(19) United States
(12) Patent Application Publication
(10) Pub. No.: US 2004/0265310 A1
(43) Pub. Date: Dec. 30, 2004

(54) Novel prostate-restricted gene expressed in prostate cancer

(76) Inventors: Daniel E. Afar, Pacific Palisades, CA (US); Rene S. Hubert, Los Angeles, CA (US); Kahan Leong, Playa del Rey, CA (US); Arthur B. Raitano, Los Angeles, CA (US); Douglas C. Saffran, Los Angeles, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/759,803

(22) Filed: Jan. 16, 2004

Related U.S. Application Data
(62) Division of application No. 09/547,788, filed on Apr. 12, 2000.
(60) Provisional application No. 60/128,860, filed on Apr. 12, 1999.

(51) Int. Cl. C12Q 1/68; C07H 21/04; A61K 39/395; C12N 9/00

(52) U.S. Cl. 424/146.1; 435/6; 435/69.1; 435/183; 435/320.1; 435/325; 530/388.26; 536/23.2

(57) ABSTRACT

A novel gene (designated 30P3C8) and its encoded protein is described. 30P3C8 exhibits restricted tissue expression in normal adult tissue and is overexpressed in prostate tissue xenografts, providing evidence that it is aberrantly expressed in at least some prostate cancers. Consequently, 30P3C8 provides a diagnostic and/or therapeutic target for prostate cancers.

This application claims the benefit of U.S. provisional application Ser. No. 60/128,860, filed Apr. 12, 1999, the entire contents of which are incorporated herein by reference.

### FIELD OF THE INVENTION

The invention described herein relates to a novel gene and its encoded protein, termed 30P3C8, and to diagnostic and therapeutic methods and compositions useful in the management of various cancers that express 30P3C8, particularly prostate cancers.

### BACKGROUND OF THE INVENTION

Cancer is the second leading cause of human death next to coronary disease. Worldwide, millions of people die from cancer every year. In the United States alone, cancer causes the death of well over a half-million people annually, with some 1.4 million new cases diagnosed per year. While deaths from heart disease have been declining significantly, those resulting from cancer generally are on the rise. In the early part of the next century, cancer is predicted to become the leading cause of death.

Worldwide, several cancers stand out as the leading killers. In particular, carcinomas of the lung, prostate, breast, colon, pancreas, and ovary represent the primary causes of cancer death. These and virtually all other carcinomas share a common lethal feature. With very few exceptions, metastatic disease from a carcinoma is fatal. Moreover, even for those cancer patients who initially survive their primary cancers, common experience has shown that their lives are dramatically altered. Many cancer patients experience strong anxieties driven by the awareness of the potential for recurrence or treatment failure. Many cancer patients experience physical debilitations following treatment. Many cancer patients experience a recurrence.

Worldwide, prostate cancer is the fourth most prevalent cancer in men. In North America and Northern Europe, it is by far the most common male cancer and is the second leading cause of cancer death in men. In the United States alone, well over 40,000 men die annually of this disease—second only to lung cancer. Despite the magnitude of these figures, there is still no effective treatment for metastatic prostate cancer. Surgical prostatectomy, radiation therapy, hormone ablation therapy, and chemotherapy continue to be the main treatment modalities. Unfortunately, these treatments are ineffective for many and are often associated with undesirable consequences.

On the diagnostic front, the lack of a prostate tumor marker that can accurately detect early-stage, localized tumors remains a significant limitation in the management of this disease. Although the serum PSA assay has been a very useful tool, its specificity and general utility is widely regarded as lacking in several important respects.

Progress in identifying additional specific markers for prostate cancer has been improved by the generation of prostate cancer xenografts that can recapitulate different stages of the disease in mice. The LAPC (Los Angeles Prostate Cancer) xenografts are prostate cancer xenografts that have survived passage in severe combined immune deficient (SCID) mice and have exhibited the capacity to mimic disease progression, including the transition from androgen dependence to androgen independence and the development of metastatic lesions (Klein et al., 1997, Nat. Med. 3:402). More recently identified prostate cancer markers include PCTA-1 (Su et al., 1996, Proc. Natl. Acad. Sci. USA 93:7252), prostate stem cell antigen (PSCA) (Reiter et al., 1998, Proc. Natl. Acad. Sci. USA 95:1735), and STEAP (Hubert et al., 1999, Proc. Natl. Acad. Sci. USA 96:14523).

While previously identified markers such as PSA, PSM, PCTA and PSCA have facilitated efforts to diagnose and treat prostate cancer, there is need for the identification of additional markers and therapeutic targets for prostate and related cancers in order to further improve diagnosis and therapy.

### SUMMARY OF THE INVENTION

The present invention relates to a novel, largely prostate-specific gene, designated 30P3C8, that is over-expressed in prostate cancer cells. 30P3C8 is also expressed in cancer cells derived from pancreas, colon, brain, bone, lung, kidney and bladder. The nucleotide and encoded amino acid sequences of a full length cDNA encoding 30P3C8 is shown in FIGS. 1A-1D (SEQ ID NOS: 1, 2). The 30P3C8 gene shows significant homology to ESTs cloned from cDNA libraries derived from a number of tissue sources, including libraries made from testis, parathyroid tumor, fetal heart and kidney. However, the 30P3C8 gene exhibits no homology to any known gene in any public database. Based on an analysis of the amino acid sequence encoded by the 30P3C8 gene, which identifies a clear consensus signal sequence, the 30P3C8 gene product appears to be a secreted protein. Analysis of tissue culture medium conditioned by cells transfected with and expressing the 30P3C8 gene product confirms that 30P3C8 protein is secreted. Moreover, western blot analysis of both whole cell lysates and supernatant from prostate cancer cells confirms that 30P3C8 protein is expressed and secreted by prostate cancer cells. The observed over-expression of 30P3C8 in prostate tumor xenografts suggests that 30P3C8 is aberrantly over-expressed in prostate cancer, and thus provides a useful diagnostic and/or therapeutic target for prostate cancers. Serum assays for the 30P3C8 gene product may be particularly useful in detecting, staging, and monitoring prostate cancer.

The invention provides polynucleotides corresponding or complementary to the 30P3C8 gene, mRNA, or fragments thereof, including cDNAs, RNAs, oligonucleotide probes, and primers. The invention further provides methods for detecting the presence of 30P3C8 polynucleotides in various biological samples. Molecular diagnostic assays for prostate cells using 30P3C8 polynucleotides are also provided. Such assays can provide diagnostic and/or prognostic information concerning the presence and degree of cancers of the prostate, pancreas, colon, brain, bone, lung, kidney and bladder. The invention further provides means for isolating cDNAs and the gene encoding 30P3C8, as well as those encoding mutated and other forms of 30P3C8. Recombinant DNA molecules containing 30P3C8 polynucleotides, cells transformed or transduced with such molecules, and host-vector systems for the expression of 30P3C8 gene products are also provided. The invention further provides 30P3C8 proteins and polypeptide fragments thereof. The invention further provides antibodies that bind to 30P3C8 proteins and polypeptide fragments thereof, including polyclonal and monoclonal antibodies, murine and other mammalian antibodies, chimeric antibodies, humanized and fully human antibodies, and antibodies labeled with a detectable marker.

The invention further provides methods for detecting the presence and status of 30P3C8 polynucleotides and proteins in various biological samples, as well as methods for identifying cells that express 30P3C8. A typical embodiment of this invention provides methods for monitoring 30P3C8 gene products in a tissue sample having or suspected of having some form of growth disregulation such as cancer.

The invention further provides various therapeutic compositions and strategies for treating cancers that express 30P3C8 such as cancer of the prostate, bladder, pancreas, colon, bone, lung, breast, testis, cervix, or ovary,30P3C8 such as prostate cancers, including therapies aimed at inhibiting the transcription, translation, processing or function of 30P3C8 as well as cancer vaccines.

### BRIEF DESCRIPTION OF THE FIGURES

FIGS. 1A-1D. Nucleotide (SEQ ID NO: 1) and deduced amino acid (SEQ ID NO: 2) sequences of 30P3C8 cDNA. The most probable START ATG and Kozak sequence are indicated in bold, and the N-terminal signal sequence is boxed.

FIG. 2A. RT-PCR analysis of 30P3C8 gene expression in prostate cancer xenografts, and other tissues and cell lines, showing expression in brain, prostate and prostate tumor xenografts. Lanes represent the following tissues: (1) brain; (2) prostate; (3) LAPC-4 AD; (4) LAPC-4 AI; (5) LAPC-9 AD; (6) LAPC-9 AI; (7) HeLa; (8) negative control.

FIG. 2B. RT-PCR analysis of 30P3C8 gene expression in normal prostate and other tissues, showing detectable expression only in normal prostate and pancreas after 25 cycles of PCR amplification. Lower level expression is detectable in a variety of other tissues after 30 cycles of amplification. Lanes represent the following tissues: Upper panel, (1) brain; (2) heart; (3) kidney; (4) liver; (5) lung; (6) pancreas; (7) placenta; (8) skeletal muscle; Lower panel, (1) colon; (2) ovary; (3) leukocytes; (4) prostate; (5) small intestine; (6) spleen; (7) testis; (8) thymus.

FIG. 3A. Northern blot analysis of 30P3C8 expression in normal tissues, showing expression of an approximately 3.5 kb transcript primarily in brain, kidney and pancreas. Lanes represent the following tissues: (1) heart; (2) brain; (3) placenta; (4) lung; (5) liver; (6) skeletal muscle; (7) kidney; (8) pancreas.

FIG. 3B. Northern blot analysis of 30P3C8 expression in normal tissues, showing substantially greater expression of an approximately 3.5 kb transcript in prostate and colon. Lanes represent the following tissues: (1) spleen; (2) thymus; (3) prostate; (4) testis; (5) ovary; (6) small intestine; (7) colon; (8) leukocytes.

FIG. 3C. Northern blot analysis of 30P3C8 expression in prostate cancer xenografts, showing overexpression of an approximately 3.5 kb transcript in all prostate cancer xenografts relative to PC-3 cells (see FIG. 3B). Lanes represent the following tissues: (1) PC-3; (2) LAPC-4 AD; (3) LAPC-4 AI; (4) LAPC-9 AD; (5) LAPC-9 AI.

FIG. 4A. High expression of 30P3C8 in prostate cancer xenografts and cancer cell lines. RNA was extracted from the LAPC xenografts and multiple cancer cell lines. Northern blots with 10 μg of total RNA/lane were probed with the 30P3C8 SSH fragment. Size standards in kilobases (kb) are indicated on the side. Lanes represent the following tissues: (1) LAPC-4 AD; (2) LAPC-4 AI; (3) LAPC-9 AD; (4) LAPC-9 AI; (5) LNCAP; (6) PC-3; (7) DU145; (8) TsuPr1; (9) LAPC-4 CL; (10) HT1197; (11) SCABER; (12) UM-UC-3; (13) TCCSUP; (14) J82; (15) 5637; (16) 293T; (17) RD-ES.

FIG. 4B. High expression of 30P3C8 in cancer cell lines. RNA was extracted from multiple cancer cell lines. Northern blots with 10 μg of total RNA/lane were probed with the 30P3C8 SSH fragment. Size standards in kilobases (kb) are indicated on the side. Lanes represent the following tissues: (18) PANC-1; (19) BxPC-3; (20) HPAC; (21) Capan-1; (22) SK-CO-1; (23) CaCo-2; (24) LoVo; (25) T84; (26) Colo-205; (27) KCL 22; (28) PFSK-1; (29) T98G; (30) SK-ES-1; (31) HOS; (32) U2-OS; (33) RD-ES; (34) CALU-1; (35) A427; (36) NCI-H82; (37) NCI-H146; (38) 769-P; (39) A498 (40) CAKI-1; (41) SW839.

FIG. 5. Expression of 30P3C8 in LAPC xenografts. RNA was extracted from LAPC xenografts that were grown subcutaneously (sc) or intra-tibially (it), within the mouse bone. Northern blots with 10 μg of total RNA/lane were probed with the 30P3C8 SSH fragment. Size standards in kilobases (kb) are indicated on the side. Lanes represent the following tissues: (1) LAPC-4 AD sc; (2) LAPC-4 AD sc; (3) LAPC-4 AD sc; (4) LAPC-4 AD it; (5) LAPC-4 AD it; (6) LAPC-4 AD it; (7) LAPC-4 AD²; (8) LAPC-9 AD sc; (9) LAPC-9 AD sc; (10) LAPC-9 AD it; (11) LAPC-9 AD it; (12) LAPC-9 AD it; (13) LAPC-3 AI sc; (14) LAPC-3 AI sc.

FIG. 6A. Expression of 30P3C8 in prostate cancer patient samples. RNA was extracted from the prostate tumors and normal adjacent tissue derived from prostate cancer patients. Northern blots with 10 μg of total RNA/lane were probed with the 30P3C8 SSH fragment. Size standards in kilobases (kb) are indicated on the side. Lanes represent the following tissues: (1) Patient 1, normal adjacent tissue; (2) Patient 1, Gleason 9 tumor; (3) Patient 2, normal adjacent tissue; (4) Patient 2, Gleason 7 tumor; (5) Patient 3, normal adjacent tissue; (6) Patient 3, Gleason 7 tumor.

FIG. 6B. Expression of 30P3C8 in prostate cancer patient samples compared to β-actin. RNA was extracted from the prostate tumors and normal adjacent tissue derived from prostate cancer patients. Northern blots with 10 μg of total RNA/lane were probed for β-actin. Lanes represent the following tissues: (1) Patient 1, normal adjacent tissue; (2) Patient 1, Gleason 9 tumor; (3) Patient 2, normal adjacent tissue; (4) Patient 2, Gleason 7 tumor; (5) Patient 3, normal adjacent tissue; (6) Patient 3, Gleason 7 tumor.

FIG. 7. Secretion of 30P3C8 protein by cells transfected with and expressing 30P3C8 cDNA.

FIG. 8A. Detection of 30P3C8 protein expression in lysates of LNCAP and LAPC4 prostate cancer cell lines by 30P3C8-specific polyclonal antibodies. LNCAP and LAPC4 cell lines were starved of androgen by incubation of cells in 2% charcoal-dextran stripped FBS for 4 days and then incubated with or without either 1 or 10 nM of the androgen analog mibolerone for 48 hours and then cells were harvested. Cell lysates (made in 2× SDS-PAGE sample buffer) were then subjected to western analysis with an affinity purified rabbit anti-peptide pAb raised to amino acids 375-389 of 30P3C8 (DVFNVEDQKRDTINL; SEQ ID NO: 30). Cell lysates (25 μg/lane) from LNCaP and LAPC4 cells, or from 293T cells as a negative control, were separated by 10-20% gradient SDS-PAGE transferred to nitrocellulose and subjected to western analysis using 2 μg/ml of affinity purified anti-30P3C8 pAb. Anti-30P3C8 immuno-reactive bands were visualized by incubation with anti-rabbit-HRP conjugated secondary antibody and enhanced chemiluminescence detection. Arrow indicates the specific 85 kD 30P3C8 band.

FIG. 8B. Detection of 30P3C8 protein expression in supernatants of LNCaP and LAPC4 prostate cancer cell lines by 30P3C8-specific polyclonal antibodies. LNCaP and LAPC4 cell lines were starved of androgen as described for FIG. 8A. Conditioned media (0.22 μM filtered) was then subjected to western analysis as described for FIG. 8A. Supernatant (20 μl) from LNCaP and LAPC4 cells, or from 293T cells as a negative control, was separated by 10-20% gradient SDS-PAGE transferred to nitrocellulose and subjected to western analysis using 2 μg/ml of affinity purified anti-30P3C8 pAb. Anti-30P3C8 immunoreactive bands were visualized by incubation with anti-rabbit-HRP conjugated secondary antibody and enhanced chemiluminescence detection. Arrow indicates the specific 85 kD 30P3C8 band.

FIG. 9. Detection of 30P3C8 protein expression in prostate cancer tissues. Tissue lysates representing LAPC4 and LAPC9 xenografts, clinical biopsy specimens representing matched normal adjacent tissue and prostate cancer tissues, whole cell lysates of LAPC4 cells, PC3 cells (androgen receptor negative), and normal prostate epithelial cells (Clonetics) were subjected to western analysis using affinity purified anti-30P3C8 pAb as described in Example 5. Arrow indicates the specific 85 kD 30P3C8 band.

### DETAILED DESCRIPTION OF THE INVENTION

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., 1989, Molecular Cloning: A Laboratory Manual, 2d ed., Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

As used herein, the terms “advanced prostate cancer”, “locally advanced prostate cancer”, “advanced disease” and “locally advanced disease” mean prostate cancers that have extended through the prostate capsule, and are meant to include stage C disease under the American Urological Association (AUA) system, stage C1-C2 disease under the Whitmore-Jewett system, and stage T3-T4 and N+ disease under the TNM (tumor, node, metastasis) system. In general, surgery is not recommended for patients with locally advanced disease, and these patients have substantially less favorable outcomes compared to patients having clinically localized (organ-confined) prostate cancer. Locally advanced disease is clinically identified by palpable evidence of induration beyond the lateral border of the prostate, or asymmetry or induration above the prostate base. Locally advanced prostate cancer is presently diagnosed pathologically following radical prostatectomy if the tumor invades or penetrates the prostatic capsule, extends into the surgical margin, or invades the seminal vesicles.

As used herein, the terms “metastatic prostate cancer” and “metastatic disease” mean prostate cancers that have spread to regional lymph nodes or to distant sites, and are meant to include stage D disease under the AUA system and stage T×N×M+ under the TNM system. As is the case with locally advanced prostate cancer, surgery is generally not indicated for patients with metastatic disease, and hormonal (androgen ablation) therapy is the preferred treatment modality. Patients with metastatic prostate cancer eventually develop an androgen-refractory state within 12 to 18 months of treatment initiation, and approximately half of these patients die within 6 months thereafter. The most common site for prostate cancer metastasis is bone. Prostate cancer bone metastases are, on balance, characteristically osteoblastic rather than osteolytic (i.e., resulting in net bone formation). Bone metastases are found most frequently in the spine, followed by the femur, pelvis, rib cage, skull and humerus. Other common sites for metastasis include lymph nodes, lung, liver and brain. Metastatic prostate cancer is typically diagnosed by open or laparoscopic pelvic lymphadenectomy, whole body radionuclide scans, skeletal radiography, and/or bone lesion biopsy.

As used herein, the term “polynucleotide” means a polymeric form of nucleotides of at least 10 bases or base pairs in length, either ribonucleotides or deoxynucleotides or a modified form of either type of nucleotide, and is meant to include single and double stranded forms of DNA.

As used herein, the term “polypeptide” means a polymer of at least 10 amino acids. Throughout the specification, standard three letter or single letter designations for amino acids are used.

As used herein, the terms “hybridize”, “hybridizing”, “hybridizes” and the like, used in the context of polynucleotides, are meant to refer to conventional hybridization conditions, preferably such as hybridization in 50% formamide/6×SSC/0.1% SDS/100 μg/ml ssDNA, in which temperatures for hybridization are above 37 degrees C. and temperatures for washing in 0.1×SSC/0.1% SDS are above 55 degrees C., and most preferably to stringent hybridization conditions.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium. citrate) and 50% formamide at 55° C., followed by a high-stringency wash consisting of 0.1×SSC containing EDTA at 55° C.

“Moderately stringent conditions” may be identified as described by Sambrook et al., 1989, Molecular Cloning: A Laboratory Manual, New York: Cold Spring Harbor Press, and include the use of washing solution and hybridization conditions (e.g., temperature, ionic strength and % SDS) less stringent than those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/mL denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

In the context of amino acid sequence comparisons, the term “identity” is used to express the percentage of amino acid residues at the same relative positions that are the same. Also in this context, the term “homology” is used to express the percentage of amino acid residues at the same relative positions that are either identical or are similar, using the conserved amino acid criteria of BLAST analysis, as is generally understood in the art. For example, % identity values may be generated by WU-BLAST-2 (Altschul et al., 1996, Methods in Enzymology 266:460-480; http://blast.wustl/edu/blast/README.html). Further details regarding amino acid substitutions, which are considered conservative under such criteria, are provided below.

Additional definitions are provided throughout the subsections that follow.

As discussed in detail below, experiments with the LAPC-4 AD xenograft in male SCID mice have resulted in the identification of genes that are involved in the progression of androgen dependent (AD) prostate cancer to androgen independent (AI) cancer. Briefly, to isolate genes that are involved in the progression of androgen dependent (AD) prostate cancer to androgen independent (AI) cancer, experiments were conducted with the LAPC-4 AD xenograft in male SCID mice. Mice that harbored LAPC-4 AD xenografts were castrated when the tumors reached a size of 1 cm in diameter. The tumors stopped growing and temporarily stopped producing the androgen dependent protein PSA. Seven to fourteen days post-castration, PSA levels were detectable again in the blood of the mice. Eventually, the tumors develop an AI phenotype and start growing again in the castrated males. Tumors were harvested at different time points after castration to identify genes that are turned on or off during the transition to androgen independence.

Suppression subtractive hybridization (SSH) (Diatchenko et al., 1996, PNAS 93:6025) was then used to identify novel genes, such as those that are overexpressed in prostate cancer, by comparing cDNAs from various androgen dependent and androgen independent LAPC xenografts. This strategy resulted in the identification of novel genes. One of these genes, designated 30P3C8, was identified from a subtraction where cDNA derived from an LAPC-4 AI tumor was subtracted from cDNA derived from an LAPC-9 AD tumor.

The 30P3C8 gene isolated using the SSH sequence as a probe encodes a secreted protein that is up-regulated in prostate cancer. The expression and secretion of 30P3C8 in prostate cancer provides a useful diagnostic and therapeutic tool for the detection and treatment of prostate cancer. In addition, 30P3C8 is expressed in cancer cells derived from pancreas, colon, brain, bone, lung, kidney and bladder, suggesting that it can be used in the detection and treatment of these cancers as well.

Structure and Expression of 30P3C8

As is further described in the Examples that follow, the 30P3C8 gene and protein have been characterized using a number of analytical approaches. For example, analyses of nucleotide coding and amino acid sequences were conducted in order to identify potentially related molecules, as well as recognizable structural domains, topological features, and other elements within the 30P3C8 mRNA and protein structures. Northern blot analyses of 30P3C8 mRNA expression was conducted in order to establish the range of normal and cancerous tissues expressing 30P3C8 message.

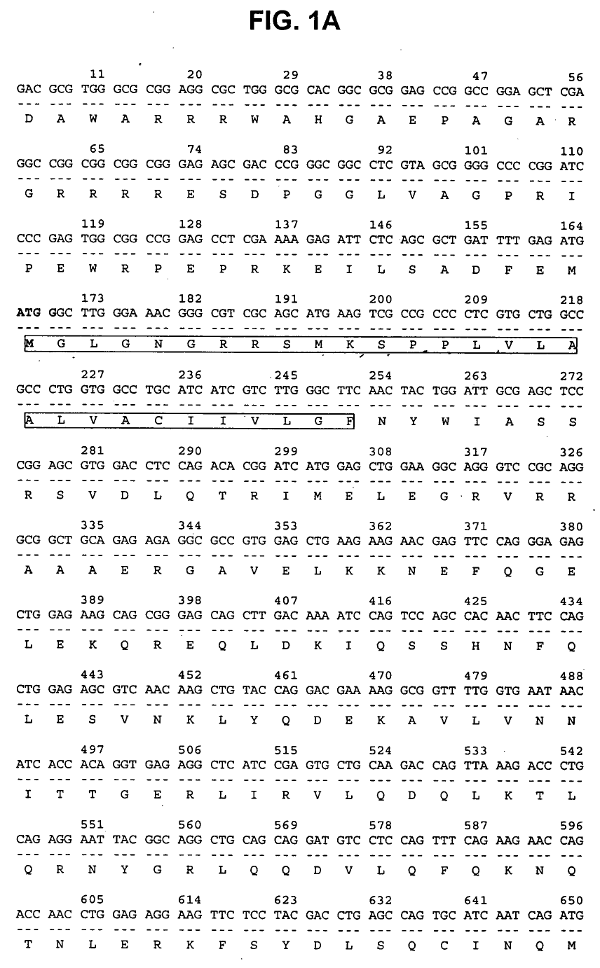

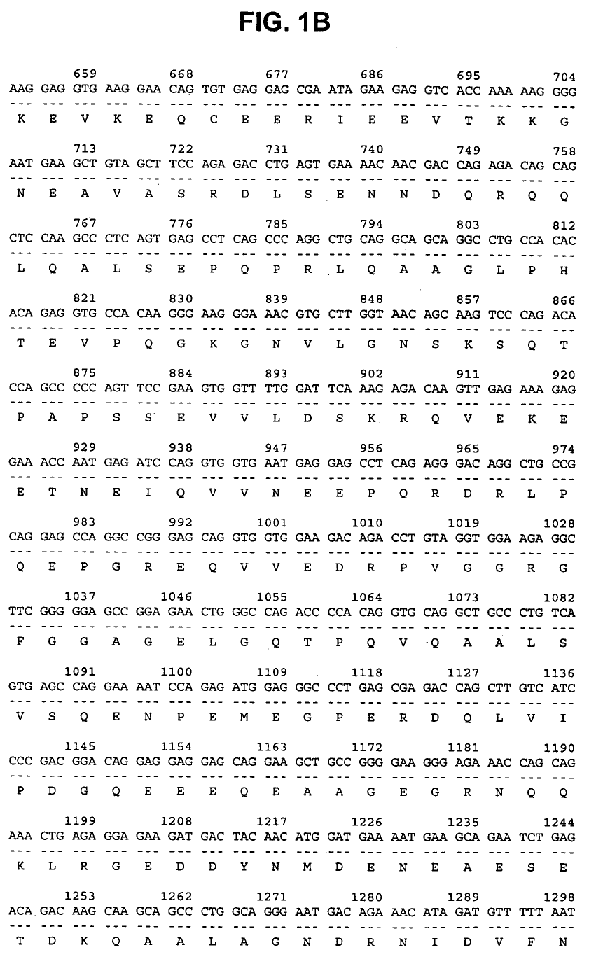

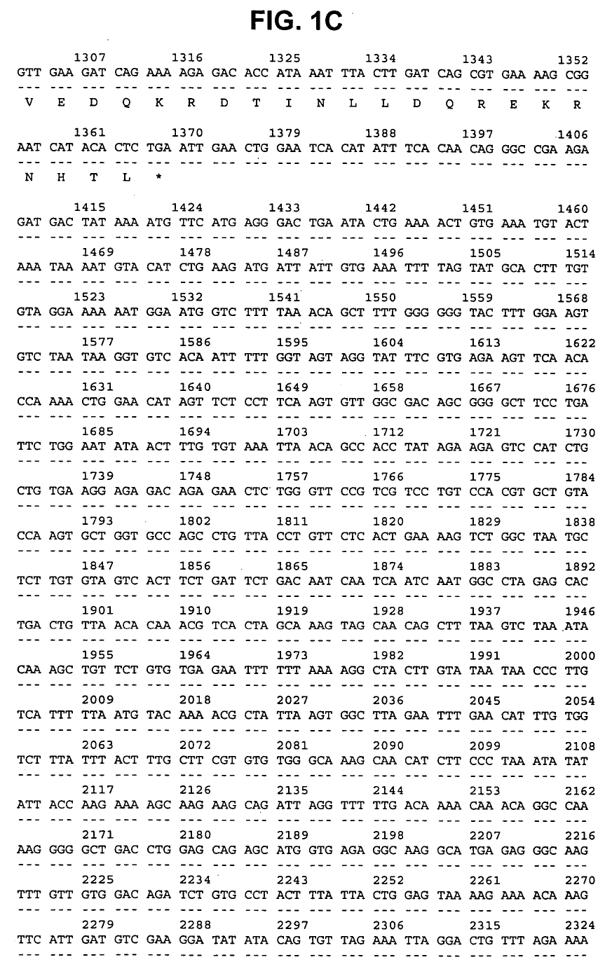

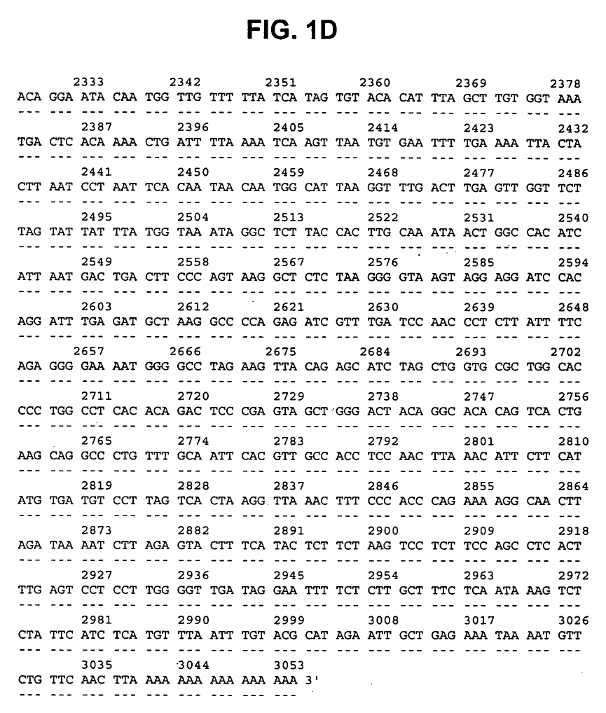

A cDNA of approximately 3 kb was isolated from a human prostate library, revealing an ORF of 400 or 401 amino acids (FIGS. 1A-1D; SEQ ID NO: 2). The protein sequence reveals an N-terminal signal sequence and a putative cleavage site at amino acid residue 28 or 29. Computer analysis of this sequence predicts that 30P3C8 is a secreted protein. In addition, the 5′ untranslated region of the 30P3C8 transcript is very GC rich (>75%), suggesting possible translational regulation of 30P3C8. The 30P3C8 cDNA sequence shows significant homology to a number of ESTs derived from a variety of sources, including testis, parathyroid tumor, fetal heart and kidney libraries. The 30P3C8 cDNA does not, however, show any significant homology to any known gene.

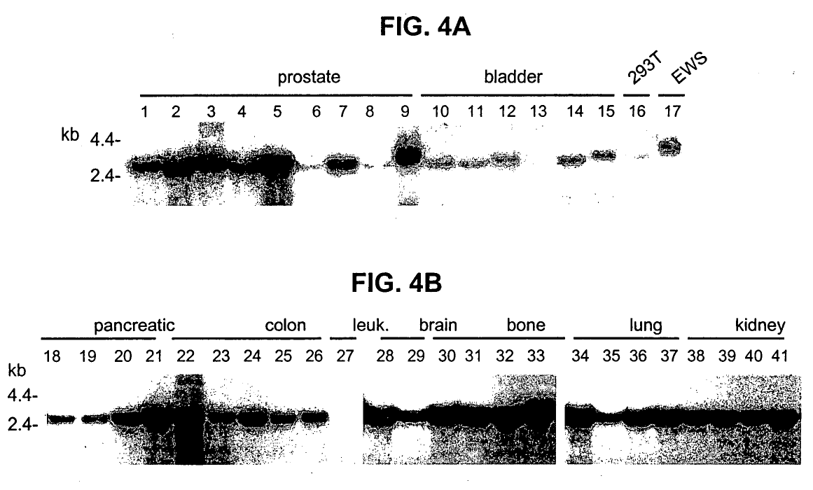

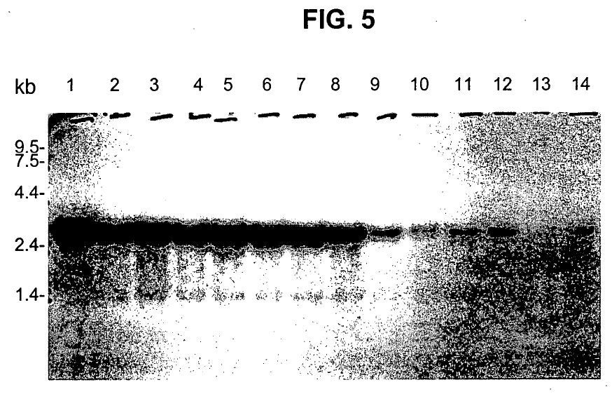

To analyze 30P3C8 expression in cancer tissues, northern blotting was performed on RNA derived from the LAPC xenografts, and several prostate and non-prostate cancer cell lines. The results show very high expression levels in LAPC-4 AD, LAPC-4 AI, LAPC-9 AD, LAPC-9 AI (FIG. 4A) and lower expression in LAPC-3 AI (FIG. 5). More detailed analysis of the xenografts shows that 30P3C8 is highly expressed in the xenografts even when grown within the tibia of mice (FIG. 5).

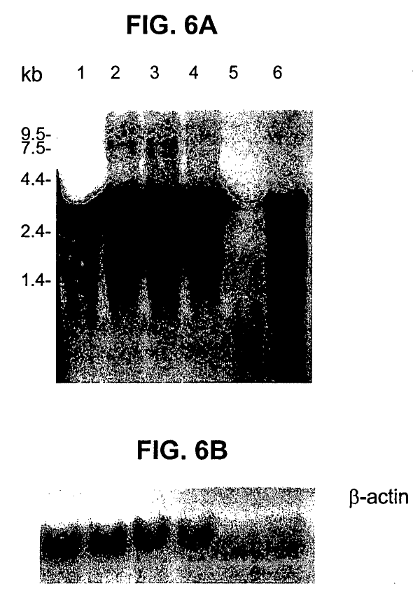

High expression levels of 30P3C8 were detected in several cancer cell lines derived from prostate (LNCaP, DU145, LAPC-4CL), pancreas (EIPAC, Capan-1), colon (SK-CO-1, CaCo-2, LoVo, T84, Colo-205), brain (PFSK-1, T98G), bone (SK-ES-1, HOS, U2-OS, RD-ES), lung (CALU-1, A427, NCI-H82, NCI-H146) and kidney (769-P, A498, CAKI-1, SW839) (FIGS. 4A-4B). Lower expression levels were also detected in multiple bladder, pancreatic and prostate cancer cell lines. Northern analysis also shows that 30P3C8 is expressed at high levels in the normal prostate and prostate tumor tissues derived from prostate cancer patients (FIG. 6A).

30P3C8 Polynucleotides

One aspect of the invention provides polynucleotides corresponding or complementary to all or part of a 30P3C8 gene, mRNA, and/or coding sequence, preferably in isolated form, including polynucleotides encoding a 30P3C8 protein and fragments thereof, DNA, RNA, DNA/RNA hybrid, and related molecules, polynucleotides or oligonucleotides complementary to a 30P3C8 gene or mRNA sequence or a part thereof, and polynucleotides or oligonucleotides that hybridize to a 30P3C8 gene, mRNA, or to a 30P3C8 encoding polynucleotide (collectively, “30P3C8 polynucleotides”). As used herein, the 30P3C8 gene and protein is meant to include the 30P3C8 genes and proteins specifically described herein and the genes and proteins corresponding to other 30P3C8 proteins and structurally similar variants of the foregoing. Such other 30P3C8 proteins and variants will generally have coding sequences that are highly homologous to the 30P3C8 coding sequence, and preferably will share at least about 50% amino acid identity and at least about 60% amino acid homology (using BLAST criteria), more preferably sharing 70% or greater homology (using BLAST criteria).

One embodiment of a 30P3C8 polynucleotide is a 30P3C8 polynucleotide having the sequence shown in FIGS. 1A-1D (SEQ ID NO: 1). A 30P3C8 polynucleotide may comprise a polynucleotide having the nucleotide sequence of human 30P3C8 as shown in FIGS. 1A-1D (SEQ ID NO: 1), wherein T can also be U; a polynucleotide that encodes all or part of the 30P3C8 protein; a sequence complementary to the foregoing; or a polynucleotide fragment of any of the foregoing. Another embodiment comprises a polynucleotide having the sequence as shown in FIGS. 1A-1D (SEQ ID NO: 1), from nucleotide residue number 165 through nucleotide residue number 1367, from residue number 165 to residue number 251 or from residue number 3 through residue number 164 or from residue number 161 through residue number 1367, wherein T can also be U. Another embodiment comprises a polynucleotide encoding a 30P3C8 polypeptide whose sequence is encoded by the cDNA contained in the plasmid p30P3C8-GTA4 as deposited with American Type Culture Collection as Designation No. 207083. Another embodiment comprises a polynucleotide that is capable of hybridizing under stringent hybridization conditions to the human 30P3C8 cDNA shown in FIGS. 1A-1D (SEQ ID NO: 1) or to a polynucleotide fragment thereof.

Typical embodiments of the invention disclosed herein include 30P3C8 polynucleotides containing specific portions of the 30P3C8 mRNA sequence (and those which are complementary to such sequences) such as those that encode the protein and fragments thereof. For example, representative embodiments of the invention disclosed herein include: polynucleotides encoding about amino acid 1 to about amino acid 10 of the 30P3C8 protein shown in FIGS. 1A-1D (SEQ ID NO: 2), polynucleotides encoding about amino acid 20 to about amino acid 30 of the 30P3C8 protein shown in FIGS. 1A-1D (SEQ ID NO: 2), polynucleotides encoding about amino acid 30 to about amino acid 40 of the 30P3C8 protein shown in FIGS. 1A-1D (SEQ ID NO: 2), polynucleotides encoding about amino acid 40 to about amino acid 50 of the 30P3C8 protein shown in FIGS. 1A-1D (SEQ ID NO: 2), polynucleotides encoding about amino acid 50 to about amino acid 60 of the 30P3C8 protein shown in FIGS. 1A-1D (SEQ ID NO: 2), polynucleotides encoding about amino acid 60 to about amino acid 70 of the 30P3C8 protein shown in FIGS. 1A-1D (SEQ ID NO: 2), polynucleotides encoding about amino acid 70 to about amino acid 80 of the 30P3C8 protein shown in FIGS. 1A-1D (SEQ ID NO: 2), polynucleotides encoding about amino acid 80 to about amino acid 90 of the 30P3C8 protein shown in FIGS. 1A-1D (SEQ ID NO: 2) and polynucleotides encoding about amino acid 90 to about amino acid 100 of the 30P3C8 protein shown in FIGS. 1A-1D (SEQ ID NO: 2), etc. Following this scheme, polynucleotides encoding portions of the amino acid sequence of amino acids 100-400 of the 30P3C8 protein are typical embodiments of the invention. Polynucleotides encoding larger portions of the 30P3C8 protein are also contemplated. For example polynucleotides encoding from about amino acid 1 (or 20 or 30 or 40 etc.) to about amino acid 20, (or 30, or 40 or 50 etc.) of the 30P3C8 protein shown in FIGS. 1A-1D (SEQ ID NO: 2) may be generated by a variety of techniques well known in the art.

Additional illustrative embodiments of 30P3C8 polynucleotides include embodiments consisting of a polynucleotide having the sequence as shown in FIGS. 1A-1D (SEQ ID NO: 1) from nucleotide residue number 1 through nucleotide residue number 500, from nucleotide residue number 500 through nucleotide residue number 1000, from nucleotide residue number 1000 through nucleotide residue number 1500, from nucleotide residue number 1500 through nucleotide residue number 2000, from nucleotide residue number 2000 through nucleotide residue number 2500 and from nucleotide residue number 2500 through nucleotide residue number 3053. These polynucleotide fragments can include any portion of the 30P3C8 sequence as shown in FIGS. 1A-1D (SEQ ID NO: 1), for example a polynucleotide having the sequence as shown in FIGS. 1A-1D (SEQ ID NO: 1) from nucleotide residue number 3 through nucleotide residue number 161 or 164, or a polynucleotide having the sequence as shown in FIGS. 1A-1D (SEQ ID NO: 1), from nucleotide residue number 162 through nucleotide residue number 1367 or the sequence from nucleotide residue number 165 through nucleotide residue number 1367.

Additional illustrative embodiments of the invention disclosed herein include 30P3C8 polynucleotide fragments encoding one or more of the biological motifs contained within the 30P3C8 protein sequence. In one embodiment, typical polynucleotide fragments of the invention can encode the signal sequence disclosed herein. In another embodiment, typical polynucleotide fragments of the invention can encode one or more of the 30P3C8 N-glycosylation sites, cAMP and cGMP-dependent protein kinase phosphorylation sites, protein kinase C phosphorylation sites, casein kinase II phosphorylation sites, tyrosine kinase phosphorylation sites, N-myristoylation sites, or amidation sites as disclosed in greater detail in the text discussing the 30P3C8 protein and polypeptides below.

The polynucleotides of the preceding paragraphs have a number of different specific uses. For example, as 30P3C8 is shown to be highly expressed in various cancers (FIGS. 4-6), these polynucleotides may be used in methods assessing the status of 30P3C8 gene products in normal versus cancerous tissues. Typically, polynucleotides encoding specific regions of the 30P3C8 protein may be used to assess the presence of perturbations (such as deletions, insertions, point mutations etc.) in specific regions of the 103P2D630P3C8 gene products. Exemplary assays include both RT-PCR assays as well as single-strand conformation polymorphism (SSCP) analysis (see, e.g., Marrogi et al., 1999, J. Cutan. Pathol. 26(8): 369-378), both of which utilize polynucleotides encoding specific regions of a protein to examine these regions within the protein.

Other specifically contemplated embodiments of the invention disclosed herein are genomic DNA, cDNAs, ribozymes, and antisense molecules, as well as nucleic acid molecules based on an alternative backbone or including alternative bases, whether derived from natural sources or synthesized. For example, antisense molecules can be RNAs or other molecules, including peptide nucleic acids (PNAs) or non-nucleic acid molecules such as phosphorothioate derivatives, that specifically bind DNA or RNA in a base pair-dependent manner. A skilled artisan can readily obtain these classes of nucleic acid molecules using the 30P3C8 polynucleotides and polynucleotide sequences disclosed herein.

Antisense technology entails the administration of exogenous oligonucleotides that bind to a target polynucleotide located within the cells. The term “antisense” refers to the fact that such oligonucleotides are complementary to their intracellular targets, e.g., 30P3C8. See for example, Jack Cohen, 1988, OLIGODEOXYNUCLEOTIDES, Antisense Inhibitors of Gene Expression, CRC Press; and Synthesis 1:1-5 (1988). The 30P3C8 antisense oligonucleotides of the present invention include derivatives such as S-oligonucleotides (phosphorothioate derivatives or S-oligos, see, Jack Cohen, supra), which exhibit enhanced cancer cell growth inhibitory action. S-oligos (nucleoside phosphorothioates) are isoelectronic analogs of an oligonucleotide (O-oligo) in which a nonbridging oxygen atom of the phosphate group is replaced by a sulfur atom. The S-oligos of the present invention may be prepared by treatment of the corresponding O-oligos with 3H-1,2-benzodithiol-3-one-1,1-dioxide, which is a sulfur transfer reagent. See Iyer, R. P. et al, 1990, J. Org. Chem. 55:4693-4698; and Iyer, R. P. et al., 1990, J. Am. Chem. Soc. 112:1253-1254, the disclosures of which are fully incorporated by reference herein.

The 30P3C8 antisense oligonucleotides of the present invention typically may be RNA or DNA that is complementary to and stably hybridizes with the first 100 N-terminal codons or last 100 C-terminal codons of the 30P3C8 genomic sequence or the corresponding mRNA. While absolute complementarity is not required, high degrees of complementarity are preferred. Use of an oligonucleotide complementary to this region allows for the selective hybridization to 30P3C8 mRNA and not to mRNA specifying other regulatory subunits of protein kinase. Preferably, the 30P3C8 antisense oligonucleotides of the present invention are a 15 to 30-mer fragment of the antisense DNA molecule having a sequence that hybridizes to 30P3C8 mRNA. Optionally, 30P3C8 antisense oligonucleotide is a 30-mer oligonucleotide that is complementary to a region in the first 10 N-terminal codons and last 10 C-terminal codons of 30P3C8. Alternatively, the antisense molecules are modified to employ ribozymes in the inhibition of 30P3C8 expression (L. A. Couture & D. T. Stinchcomb, 1996, Trends Genet. 12: 510-515).

Further specific embodiments of this aspect of the invention include primers and primer pairs, which allow the specific amplification of the polynucleotides of the invention or of any specific parts thereof, and probes that selectively or specifically hybridize to nucleic acid molecules of the invention or to any part thereof. Probes may be labeled with a detectable marker, such as, for example, a radioisotope, fluorescent compound, bioluminescent compound, a chemiluminescent compound, metal chelator or enzyme. Such probes and primers can be used to detect the presence of a 30P3C8 polynucleotide in a sample and as a means for detecting a cell expressing a 30P3C8 protein.

Examples of such probes include polypeptides comprising all or part of the human 30P3C8 cDNA sequences shown in FIGS. 1A-1D (SEQ ID NO: 1). Examples of primer pairs capable of specifically amplifying 30P3C8 mRNAs are also described in the Examples that follow. As will be understood by the skilled artisan, a great many different primers and probes may be prepared based on the sequences provided herein and used effectively to amplify and/or detect a 30P3C8 mRNA.

As used herein, a polynucleotide is said to be “isolated” when it is substantially separated from contaminant polynucleotides that correspond or are complementary to genes other than the 30P3C8 gene or that encode polypeptides other than 30P3C8 gene product or fragments thereof. A skilled artisan can readily employ nucleic acid isolation procedures to obtain an isolated 30P3C8 polynucleotide.

The 30P3C8 polynucleotides of the invention are useful for a variety of purposes, including but not limited to their use as probes and primers for the amplification and/or detection of the 30P3C8 gene(s), mRNA(s), or fragments thereof; as reagents for the diagnosis and/or prognosis of prostate cancer and other cancers; as coding sequences capable of directing the expression of 30P3C8 polypeptides; as tools for modulating or inhibiting the expression of the 30P3C8 gene(s) and/or translation of the 30P3C8 transcript(s); and as therapeutic agents.

Isolation of 30P3C8-Encoding Nucleic Acid Molecules

The 30P3C8 cDNA sequences described herein enable the isolation of other polynucleotides encoding 30P3C8 gene product(s), as well as the isolation of polynucleotides encoding 30P3C8 gene product homologs, alternatively spliced isoforms, allelic variants, and mutant forms of the 30P3C8 gene product. Various molecular cloning methods that can be employed to isolate full length cDNAs encoding a 30P3C8 gene are well known (See, e.g., Sambrook, J. et al., 1989, Molecular Cloning: A Laboratory Manual, 2d ed., Cold Spring Harbor Press, New York; Ausubel et al., eds., 1995, Current Protocols in Molecular Biology, Wiley and Sons). For example, lambda phage cloning methodologies may be conveniently employed, using commercially available cloning systems (e.g., Lambda ZAP Express, Stratagene). Phage clones containing 30P3C8 gene cDNAs may be identified by probing with a labeled 30P3C8 cDNA or a fragment thereof. For example, in one embodiment, the 30P3C8 cDNA (FIGS. 1A-1D; SEQ ID NO: 1) or a portion thereof can be synthesized and used as a probe to retrieve overlapping and full length cDNAs corresponding to a 30P3C8 gene. The 30P3C8 gene itself may be isolated by screening genomic DNA libraries, bacterial artificial chromosome libraries (BACs), yeast artificial chromosome libraries (YACs), and the like, with 30P3C8 DNA probes or primers.

Recombinant DNA Molecules and Host-Vector Systems

The invention also provides recombinant DNA or RNA molecules containing a 30P3C8 polynucleotide, including but not limited to phages, plasmids, phagemids, cosmids, YACs, BACs, as well as various viral and non-viral vectors well known in the art, and cells transformed or transfected with such recombinant DNA or RNA molecules. As used herein, a recombinant DNA or RNA molecule is a DNA or RNA molecule that has been subjected to molecular manipulation in vitro. Methods for generating such molecules are well known (see, e.g., Sambrook et al, 1989, supra).

The invention further provides a host-vector system comprising a recombinant DNA molecule containing a 30P3C8 polynucleotide within a suitable prokaryotic or eukaryotic host cell. Examples of suitable eukaryotic host cells include a yeast cell, a plant cell, or an animal cell, such as a mammalian cell or an insect cell (e.g., a baculovirus-infectible cell such as an Sf9 or HighFive cell). Examples of suitable mammalian cells include various prostate cancer cell lines such as PrEC, LNCaP and TsuPr1, other transfectable or transducible prostate cancer cell lines, as well as a number of mammalian cells routinely used for the expression of recombinant proteins (e.g., COS, CHO, 293, 293T cells). More particularly, a polynucleotide comprising the coding sequence of 30P3C8 may be used to generate 30P3C8 proteins or fragments thereof using any number of host-vector systems routinely used and widely known in the art.

A wide range of host-vector systems suitable for the expression of 30P3C8 proteins or fragments thereof are available (see, e.g., Sambrook et al., 1989, supra; Current Protocols in Molecular Biology, 1995, supra). Preferred vectors for mammalian expression include but are not limited to pcDNA 3.1 myc-His-tag (Invitrogen) and the retroviral vector pSRcctkneo (Muller et al., 1991, MCB 11:1785). Using these expression vectors, 30P3C8 may be preferably expressed in several prostate cancer and non-prostate cell lines, including for example 293, 293T, rat-1, NIH 3T3 and TsuPr1. The host-vector systems of the invention are useful for the production of a 30P3C8 protein or fragment thereof. Such host-vector systems may be employed to study the functional properties of 30P3C8 and 30P3C8 mutations.

Recombinant human 30P3C8 protein may be produced by mammalian cells transfected with a construct encoding 30P3C8. In an illustrative embodiment described in the Examples, 293T cells can be transfected with an expression plasmid encoding 30P3C8, the 30P3C8 protein is expressed in the 293T cells, and the recombinant 30P3C8 protein can be isolated using standard purification methods (e.g., affinity purification using anti-30P3C8 antibodies). In another embodiment, also described in the Examples herein, the 30P3C8 coding sequence is subcloned into the retroviral vector pSRaMSVtkneo and used to infect various mammalian cell lines, such as NIH 3T3, TsuPr1, 293 and rat-1 in order to establish 30P3C8 expressing cell lines. Various other expression systems well known in the art may also be employed. Expression constructs encoding a leader peptide joined in frame to the 30P3C8 coding sequence may be used for the generation of a secreted form of recombinant 30P3C8 protein.

Proteins encoded by the 30P3C8 genes, or by fragments thereof, will have a variety of uses, including but not limited to generating antibodies and in methods for identifying ligands and other agents and cellular constituents that bind to a 30P3C8 gene product. Antibodies raised against a 30P3C8 protein or fragment thereof may be useful in diagnostic and prognostic assays, and imaging methodologies in the management of human cancers characterized by expression of 30P3C8 protein, including but not limited to cancers of the prostate, pancreas, colon, brain, bone, lung, kidney, and bladder. Such antibodies may be expressed intracellularly and used in methods of treating patients with such cancers. Various immunological assays useful for the detection of 30P3C8 proteins are contemplated, including but not limited to various types of radioimmunoassays, enzyme-linked immunosorbent assays (ELISA), enzyme-linked immunofluorescent assays (ELIFA), immunocytochemical methods, and the like. Such antibodies may be labeled and used as immunological imaging reagents capable of detecting 30P3C8 expressing cells (e.g., in radioscintigraphic imaging methods). 30P3C8 proteins may also be particularly useful in generating cancer vaccines, as further described below. 30P3C8 Polypeptides

Another aspect of the present invention provides 30P3C8 proteins and polypeptide fragments thereof. The 30P3C8 proteins of the invention include those specifically identified herein, as well as allelic variants, conservative substitution variants and homologs that can be isolated/generated and characterized without undue experimentation following the methods outlined below. Fusion proteins that combine parts of different 30P3C8 proteins or fragments thereof, as well as fusion proteins of a 30P3C8 protein and a heterologous polypeptide are also included. Such 30P3C8 proteins will be collectively referred to as the 30P3C8 proteins, the proteins of the invention, or 30P3C8. As used herein, the term “30P3C8 polypeptide” refers to a polypeptide fragment or a 30P3C8 protein of at least 10 amino acids, preferably at least 15 amino acids.

Specific embodiments of 30P3C8 proteins comprise a polypeptide having the amino acid sequence of human 30P3C8 as shown in FIGS. 1A-1D (SEQ ID NO: 2). Alternatively, embodiments of 30P3C8 proteins comprise variant polypeptides having alterations in the amino acid sequence of human 30P3C8 as shown in FIGS. 1A-1D (SEQ ID NO: 2).

In general, naturally occurring allelic variants of human 30P3C8 will share a high degree of structural identity and homology (e.g., 90% or more identity). Typically, allelic variants of the 30P3C8 proteins will contain conservative amino acid substitutions within the 30P3C8 sequences described herein or will contain a substitution of an amino acid from a corresponding position in a 30P3C8 homologue. One class of 30P3C8 allelic variants will be proteins that share a high degree of homology with at least a small region of a particular 30P3C8 amino acid sequence, but will further contain a radical departure from the sequence, such as a non-conservative substitution, truncation, insertion or frame shift.

Conservative amino acid substitutions can frequently be made in a protein without altering either the conformation or the function of the protein. Such changes include substituting any of isoleucine (I), valine (V), and leucine (L) for any other of these hydrophobic amino acids; aspartic acid () for glutamic acid (E) and vice versa; glutamine (Q) for asparagine (N) and vice versa; and serine (S) for threonine (C) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (M). Methionine (M), which is relatively hydrophobic, can frequently be interchanged with leucine and isoleucine, and sometimes with valine. Lysine (K) and arginine. (R) are frequently interchangeable in locations in which the significant feature of the amino acid residue is its charge and the differing pK's of these two amino acid residues are not significant. Still other changes can be considered “conservative” in particular environments.

Embodiments of the invention disclosed herein include a wide variety of art accepted variants of 30P3C8 proteins such as polypeptides having amino acid insertions, deletions and substitutions. 30P3C8 variants can be made using methods known in the art such as site-directed mutagenesis, alanine scanning, and PCR mutagenesis. Site-directed mutagenesis (Carter et al., 1986, Nucl. Acids Res. 13:4331; Zoller et al., 1987, Nucl. Acids Res. 10:6487), cassette mutagenesis (Wells et al., 1985, Gene 34:315), restriction selection mutagenesis (Wells et al., 1986, Philos. Trans. R. Soc. London Ser. A, 317:415) or other known techniques can be performed on the cloned DNA to produce the 30P3C8 variant DNA. Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant. Alanine is also typically preferred because it is the most common amino acid. Further, it is frequently found in both buried and exposed positions (Creighton, The Proteins, (W.H. Freeman & Co., N.Y.); Chothia, 1976, J. Mol. Biol., 150:1). If alanine substitution does not yield adequate amounts of variant, an isosteric amino acid can be used.

As discussed above, embodiments of the claimed invention include polypeptides containing less than the 400 (or 401) amino acid sequence of the 30P3C8 protein shown in FIGS. 1A-1D (SEQ ID NO: 2) (and the polynucleotides encoding such polypeptides). For example, representative embodiments of the invention disclosed herein include polypeptides consisting of about amino acid 1 to about amino acid 10 of the 30P3C8 protein shown in FIGS. 1A-1D (SEQ ID NO: 2), polypeptides consisting of about amino acid 20 to about amino acid 30 of the 30P3C8 protein shown in FIGS. 1A-1D (SEQ ID NO: 2), polypeptides consisting of about amino acid 30 to about amino acid 40 of the 30P3C8 protein shown in FIGS. 1A-1D (SEQ ID NO: 2), polypeptides consisting of about amino acid 40 to about amino acid 50 of the 30P3C8 protein shown in FIGS. 1A-1D (SEQ ID NO: 2), polypeptides consisting of about amino acid 50 to about amino acid 60 of the 30P3C8 protein shown in FIGS. 1A-1D (SEQ ID NO: 2), polypeptides consisting of about amino acid 60 to about amino acid 70 of the 30P3C8 protein shown in FIGS. 1A-1D (SEQ ID NO: 2), polypeptides consisting of about amino acid 70 to about amino acid 80 of the 30P3C8 protein shown in FIGS. 1A-1D (SEQ ID NO: 2), polypeptides consisting of about amino acid 80 to about amino acid 90 of the 30P3C8 protein shown in FIGS. 1A-1D (SEQ ID NO: 2) and polypeptides consisting of about amino acid 90 to about amino acid 100 of the 30P3C8 protein shown in FIGS. 1A-1D (SEQ ID NO: 2), etc. Following this scheme, polypeptides consisting of portions of the amino acid sequence of amino acids 100-400 of the 30P3C8 protein are typical embodiments of the invention. Polypeptides consisting of larger portions of the 30P3C8 protein are also contemplated. For example polypeptides consisting of about amino acid 1 (or 20 or 30 or 40 etc.) to about amino acid 20, (or 30, or 40 or 50 etc.) of the 30P3C8 protein shown in FIGS. 1A-1D (SEQ ID NO: 2) may be generated by a variety of techniques well known in the art.

Additional illustrative embodiments of the invention disclosed herein include 30P3C8 polypeptides containing the amino acid residues of one or more of the biological motifs contained within the 30P3C8 polypeptide sequence as shown in FIGS. 1A-1D (SEQ ID NO: 2). In one embodiment, typical polypeptides of the invention can contain the 30P3C8 signal sequence at residues 1 through 28 or 29. In another embodiment, typical polypeptides of the invention can contain one or more of the 30P3C8 N-glycosylation sites such as NITT (SEQ ID NO: 3) at residues 108-111, NQTN (SEQ ID NO: 4) at residues 143-146, and/or NHTL (SEQ ID NO: 5) at residues 397-400. In another embodiment, typical polypeptides of the invention can contain one or more of the 30P3C8 cAMP- and cGMP-dependent protein kinase phosphorylation sites such as RKFS (SEQ ID NO: 6) at residues 149-152, and/or KRDT (SEQ ID NO: 7) at residues 383-386. In another embodiment, typical polypeptides of the invention can contain one or more of the 30P3C8 protein kinase C phosphorylation sites such as SMK at residues 9-11, SSR at residues 35-37, TKK at residues 177-179, SKR at residues 245-247 and/or TDK at residues 361-363. In another embodiment, typical polypeptides of the invention can contain one or more of the 30P3C8 casein kinase II phosphorylation sites such as TTGE (SEQ ID NO: 8) at residues 110-113, TNLE (SEQ ID NO: 9) at residues 145-148, and/or SETD (SEQ ID NO: 10) at residues 359-362. In another embodiment, typical polypeptides of the invention can contain a tyrosine kinase phosphorylation site such as KLRGEDDY (SEQ ID NO: 11) at residues 343-350. In another embodiment, typical polypeptides of the invention can contain one or more of the N-myristoylation sites such as GLGNGR (SEQ ID NO: 12) at residues 2-7, GLPHTE (SEQ ID NO: 13) at residues 213-218, GNVLGN (SEQ ID NO: 15) at residues 224-229, GNSKSQ (SEQ ID NO: 15) at residues 228-233, and/or GNDRNI (SEQ ID NO: 16) at residues 369-374. In another embodiment, typical polypeptides of the invention can contain an amidation sites such as NGRR (SEQ ID NO: 17) at residues 5-8. Related embodiments of these inventions include polypeptides containing combinations of the different motifs discussed above with preferable embodiments being those which contain no insertions, deletions or substitutions either within the motifs or the intervening sequences of these polypeptides.

Illustrative examples of such embodiments includes a polypeptide having one or more amino acid sequences selected from the group consisting of NITT (SEQ ID NO: 3), NQTN (SEQ ID NO: 4), NHTL (SEQ ID NO: 5), RKFS (SEQ ID NO: 6), KRDT (SEQ ID NO: 7), SMK, SSR, TKK, SKR, TDK, TTGE (SEQ ID NO: 8), TNLE (SEQ ID NO: 9), SETD (SEQ ID NO: 10), KLRGEDDY (SEQ ID NO: 11), GLGNGR (SEQ ID NO: 12), GLPHTE (SEQ ID NO: 13), GNVLGN (SEQ ID NO: 14), GNSKSQ (SEQ ID NO: 15), GNDRNI (SEQ ID NO: 16), and NGRR (SEQ ID NO: 17). In a preferred embodiments, the polypeptide includes two three or four or five or six or more amino acid sequences selected from the group consisting of NITT (SEQ ID NO: 3), NQTN (SEQ ID NO: 4), NHTL (SEQ ID NO: 5), RKFS (SEQ ID NO: 6), KRDT (SEQ ID NO: 7), SMK, SSR, TKK, SKR, TDK, TTGE (SEQ ID NO: 8), TNLE (SEQ ID NO: 9), SETD (SEQ ID NO: 10), KLRGEDDY (SEQ ID NO: 11), GLGNGR (SEQ ID NO: 12), GLPHTE (SEQ ID NO: 13), GNVLGN (SEQ ID NO: 14), GNSKSQ (SEQ ID NO: 15), GNDRNI (SEQ ID NO: 16), and NGRR (SEQ ID NO: 17). Alternatively polypeptides having other combinations of the biological motifs disclosed herein are also contemplated.

In yet another embodiment of the invention, typical polypeptides can contain amino acid sequences that are unique to one or more 30P3C8 alternative splicing variants. The monitoring of alternative splice variants of 30P3C8 is useful because changes in the alternative splicing of proteins is suggested as one of the steps in a series of events that lead to the progression of cancers (see e.g. Carstens et al., 1997, Oncogene 15(25):3059-3065). Consequently, monitoring of alternative splice variants of 30P3C8 provides an additional means to evaluate syndromes associated with perturbations in 30P3C8 gene products such as cancers.

Polypeptides consisting of one or more of the 30P3C8 motifs discussed above are useful in elucidating the specific characteristics of a malignant phenotype in view of the observation that the 30P3C8 motifs discussed above are associated with growth disregulation and because 30P3C8 is overexpressed in cancers (FIGS. 4-6). Casein kinase II, cAMP and cCMP-dependent protein kinase and protein kinase C for example are enzymes known to be associated with the development of the malignant phenotype (see e.g. Chen et al., 1998, Lab Invest., 78(2):165-174; Gaiddon et al., 1995, Endocrinology 136(10):4331-4338; Hall et al., 1996, Nucleic Acids Research 24(6):1119-1126; Peterziel et al., 1999, Oncogene 18(46):6322-6329; and O'Brian, 1998, Oncol. Rep. 5(2): 305-309). Moreover, both glycosylation and myristoylation are protein modifications also associated with cancer and cancer progression (see e.g. Dennis et al., 1999, Biochim. Biophys. Acta 1473(1):21-34; Raju et al., 1997, Exp. Cell Res. 235(1):145-154).

The polypeptides of the preceding paragraphs have a number of different specific uses. As 30P3C8 is shown to be highly expressed in prostate, pancreatic, colon, brain, bone, lung, kidney and bladder cancers (FIGS. 4-6), these polypeptides may be used in methods assessing the status of 30P3C8 gene products in normal versus cancerous tissues and elucidating the malignant phenotype. Typically, polypeptides encoding specific regions of the 30P3C8 protein may be used to assess the presence of perturbations (such as deletions, insertions, point mutations etc.) in specific regions of the 30P3C8 gene products. Exemplary assays can utilize antibodies targeting a 30P3C8 polypeptides containing the amino acid residues of one or more of the biological motifs contained within the 30P3C8 polypeptide sequence in order to evaluate the characteristics of this region in normal versus cancerous tissues. Alternatively, 30P3C8 polypeptides containing the amino acid residues of one or more of the biological motifs contained within the 30P3C8 polypeptide sequence can be used to screen for factors that interact with that region of 30P3C8.

As discussed above, redundancy in the genetic code permits variation in 30P3C8 gene sequences. In particular, one skilled in the art will recognize specific codon preferences by a specific host species and can adapt the disclosed sequence as preferred for a desired host. For example, preferred codon sequences typically have rare codons (i.e., codons having a usage frequency of less than about 20% in known sequences of the desired host) replaced with higher frequency codons. Codon preferences for a specific organism may be calculated, for example, by utilizing codon usage tables available on the Internet at the following address: http://www.dna.affrc.go.jp/˜nakamura/codon.html. Nucleotide sequences that have been optimized for a particular host species by replacing any codons having a usage frequency of less than about 20% are referred to herein as “codon optimized sequences.

Additional sequence modifications are known to enhance protein expression in a cellular host. These include elimination of sequences encoding spurious polyadenylation signals, exon/intron splice site signals, transposon-like repeats, and/or other such well-characterized sequences that may be deleterious to gene expression. The GC content of the sequence may be adjusted to levels average for a given cellular host, as calculated by reference to known genes expressed in the host cell. Where possible, the sequence may also be modified to avoid predicted hairpin secondary mRNA structures. Other useful modifications include the addition of a translational initiation consensus sequence at the start of the open reading frame, as described in Kozak, 1989, Mol. Cell Biol., 9:5073-5080. Nucleotide sequences that have been optimized for expression in a given host species by elimination of spurious polyadenylation sequences, elimination of exon/intron splicing signals, elimination of transposon-like repeats and/or optimization of GC content in addition to codon optimization are referred to herein as an “expression enhanced sequence.”

30P3C8 proteins may be embodied in many forms, preferably in isolated form. As used herein, a protein is said to be “isolated” when physical, mechanical or chemical methods are employed to remove the 30P3C8 protein from cellular constituents that are normally associated with the protein. A skilled artisan can readily employ standard purification methods to obtain an isolated 30P3C8 protein. A purified 30P3C8 protein molecule will be substantially free of other proteins or molecules that impair the binding of 30P3C8 to antibody or other ligand. The nature and degree of isolation and purification will depend on the intended use. Embodiments of a 30P3C8 protein include a purified 30P3C8 protein and a functional, soluble 30P3C8 protein. In one form, such functional, soluble 30P3C8 proteins or fragments thereof retain the ability to bind antibody or other ligand.

The invention also provides 30P3C8 polypeptides comprising biologically active fragments of the 30P3C8 amino acid sequence, such as a polypeptide corresponding to part of the amino acid sequence for 30P3C8 as shown in FIGS. 1A-1D (SEQ ID NO: 2). Such polypeptides of the invention exhibit properties of the 30P3C8 protein, such as the ability to elicit the generation of antibodies that specifically bind an epitope associated with the 30P3C8 protein. 30P3C8 polypeptides can be generated using standard peptide synthesis technology or using chemical cleavage methods well known in the art based on the amino acid sequences of the human 30P3C8 proteins disclosed herein. Alternatively, recombinant methods can be used to generate nucleic acid molecules that encode a polypeptide fragment of a 30P3C8 protein. In this regard, the 30P3C8-encoding nucleic acid molecules described herein provide means for generating defined fragments of 30P3C8 proteins. 30P3C8 polypeptides are particularly useful in generating and characterizing domain specific antibodies (e.g., antibodies recognizing an extracellular or intracellular epitope of a 30P3C8 protein), in identifying agents or cellular factors that bind to 30P3C8 or a particular structural domain thereof, and in various therapeutic contexts, including but not limited to cancer vaccines.

30P3C8 polypeptides containing particularly interesting structures can be predicted and/or identified using various analytical techniques well known in the art, including, for example, the methods of Chou-Fasman, Garnier-Robson, Kyte-Doolittle, Eisenberg, Karplus-Schultz or Jameson-Wolf analysis, or on the basis of immunogenicity. Fragments containing such structures are particularly useful in generating subunit specific anti-30P3C8 antibodies or in identifying cellular factors that bind to 30P3C8.

In an embodiment described in the examples that follow, 30P3C8 can be conveniently expressed in cells (such as 293T cells) transfected with a commercially available expression vector such as a CMV-driven expression vector encoding 30P3C8 with a C-terminal 6×His and MYC tag (pcDNA3.1/mycHIS, Invitrogen or Tag5, GenHunter Corporation, Nashville Tenn.). The Tag5 vector provides an IgGK secretion signal that can be used to facilitate the production of a secreted 30P3C8 protein in transfected cells. The secreted HIS-tagged 30P3C8 in the culture media may be purified using a nickel column using standard techniques.

Modifications of 30P3C8 such as covalent modifications are included within the scope of this invention. One type of covalent modification includes reacting targeted amino acid residues of a 30P3C8 polypeptide with an organic derivatizing agent that is capable of reacting with selected side chains or the N— or C-terminal residues of the 30P3C8. Another type of covalent modification of the 30P3C8 polypeptide included within the scope of this invention comprises altering the native glycosylation pattern of the polypeptide. “Altering the native glycosylation pattern” is intended for purposes herein to mean deleting one or more carbohydrate moieties found in native sequence 30P3C8 (either by removing the underlying glycosylation site or by deleting the glycosylation by chemical and/or enzymatic means), and/or adding one or more glycosylation sites that are not present in the native sequence 30P3C8. In addition, the phrase includes qualitative changes in the glycosylation of the native proteins, involving a change in the nature and proportions of the various carbohydrate moieties present. Another type of covalent modification of 30P3C8 comprises linking the 30P3C8 polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

The 30P3C8 of the present invention may also be modified in a way to form a chimeric molecule comprising 30P3C8 fused to another, heterologous polypeptide or amino acid sequence. In one embodiment, such a chimeric molecule comprises a fusion of the 30P3C8 with a polyhistidine epitope tag, which provides an epitope to which immobilized nickel can selectively bind. The epitope tag is generally placed at the amino- or carboxyl-terminus of the 30P3C8. In an alternative embodiment, the chimeric molecule may comprise a fusion of the 30P3C8 with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of a 30P3C8 polypeptide in place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130 issued Jun. 27, 1995.

30P3C8 Antibodies

The term “antibody” is used in the broadest sense and specifically covers single anti-30P3C8 monoclonal antibodies (including agonist, antagonist and neutralizing antibodies) and anti-30P3C8 antibody compositions with polyepitopic specificity. The term “monoclonal antibody” (mAb) as used herein refers to an antibody obtained from a population of substantially homogeneous antibodies, i.e. the antibodies comprising the individual population are identical except for possible naturally-occurring mutations that may be present in minor amounts.

Another aspect of the invention provides antibodies that bind to 30P3C8 proteins and polypeptides. The most preferred antibodies will specifically bind to a 30P3C8 protein and will not bind (or will bind weakly) to non-30P3C8 proteins and polypeptides. Anti-30P3C8 antibodies that are particularly contemplated include monoclonal and polyclonal antibodies as well as fragments containing the antigen binding domain and/or one or more complementarity determining regions of these antibodies. As used herein, an antibody fragment is defined as at least a portion of the variable region of the immunoglobulin molecule that binds to its target, i.e., the antigen binding region.

30P3C8 antibodies of the invention may be particularly useful in prostate cancer diagnostic and prognostic assays, and imaging methodologies. Intracellularly expressed antibodies (e.g., single chain antibodies) may be therapeutically useful in treating cancers in which the expression of 30P3C8 is involved, such as for example advanced and metastatic prostate cancers. 30P3C8 antibodies can be used for delivery of a toxin or therapeutic molecule. Such delivery of a toxin or therapeutic molecule can be achieved using known methods of conjugating a second molecule to the 30P3C8 antibody or fragment thereof. Similarly, such antibodies may be useful in the treatment, diagnosis, and/or prognosis of other cancers, to the extent 30P3C8 is also expressed or overexpressed in other types of cancer, such as pancreatic, colon, brain, bone, lung, kidney and bladder cancers.

The invention also provides various immunological assays useful for the detection and quantification of 30P3C8 and mutant 30P3C8 proteins and polypeptides. Such assays generally comprise one or more 30P3C8 antibodies capable of recognizing and binding a 30P3C8 or mutant 30P3C8 protein, as appropriate, and may be performed within various immunological assay formats well known in the art, including but not limited to various types of radioimmunoassays, enzyme-linked immunosorbent assays (ELISA), enzyme-linked immunofluorescent assays (ELIFA), and the like. In addition, immunological imaging methods capable of detecting prostate cancer and other cancers expressing 30P3C8 are also provided by the invention, including but limited to radioscintigraphic imaging methods using labeled 30P3C8 antibodies. Such assays may be clinically useful in the detection, monitoring, and prognosis of 30P3C8 expressing cancers such as prostate, pancreatic, colon, brain, bone, lung, kidney and bladder cancers.

30P3C8 antibodies may also be used in methods for purifying 30P3C8 and mutant 30P3C8 proteins and polypeptides and for isolating 30P3C8 homologues and related molecules. For example, in one embodiment, the method of purifying a 30P3C8 protein comprises incubating a 30P3C8 antibody, which has been coupled to a solid matrix, with a lysate or other solution containing 30P3C8 under conditions that permit the 30P3C8 antibody to bind to 30P3C8; washing the solid matrix to eliminate impurities; and eluting the 30P3C8 from the coupled antibody. Other uses of the 30P3C8 antibodies of the invention include generating anti-idiotypic antibodies that mimic the 30P3C8 protein.

Various methods for the preparation of antibodies are well known in the art. For example, antibodies may be prepared by immunizing a suitable mammalian host using a 30P3C8 protein, peptide, or fragment, in isolated or immunoconjugated form (Harlow, and Lane, eds., 1988, Antibodies: A Laboratory Manual, CSH Press; Harlow, 1989, Antibodies, Cold Spring Harbor Press, N.Y.). In addition, fusion proteins of 30P3C8 may also be used, such as a 30P3C8 GST-fusion protein. In a particular embodiment, a GST fusion protein comprising all or most of the open reading frame amino acid sequence of FIGS. 1A-1D (SEQ ID NO: 2) may be produced and used as an immunogen to generate appropriate antibodies. In another embodiment, a 30P3C8 peptide may be synthesized and used as an immunogen.

In addition, naked DNA immunization techniques known in the art may be used (with or without purified 30P3C8 protein or 30P3C8 expressing cells) to generate an immune response to the encoded immunogen (for review, see Donnelly et al., 1997, Ann. Rev. Immunol. 15:617-648).

The amino acid sequence of the 30P3C8 as shown in FIGS. 1A-1D (SEQ ID NO: 2) may be used to select specific regions of the 30P3C8 protein for generating antibodies. For example, hydrophobicity and hydrophilicity analyses of the 30P3C8 amino acid sequence may be used to identify hydrophilic regions in the 30P3C8 structure. Regions of the 30P3C8 protein that show immunogenic structure, as well as other regions and domains, can readily be identified using various other methods known in the art, such as Chou-Fasman, Garnier-Robson, Kyte-Doolittle, Eisenberg, Karplus-Schultz or Jameson-Wolf analysis. Methods for the generation of 30P3C8 antibodies are further illustrated by way of the examples provided herein.

Methods for preparing a protein or polypeptide for use as an immunogen and for preparing immunogenic conjugates of a protein with a carrier such as BSA, KLH, or other carrier proteins are well known in the art. In some circumstances, direct conjugation using, for example, carbodiimide reagents may be used; in other instances linking reagents such as those supplied by Pierce Chemical Co., Rockford, Ill., may be effective. Administration of a 30P3C8 immunogen is conducted generally by injection over a suitable time period and with use of a suitable adjuvant, as is generally understood in the art. During the immunization schedule, titers of antibodies can be taken to determine adequacy of antibody formation.

30P3C8 monoclonal antibodies may be produced by various means well known in the art. For example, immortalized cell lines that secrete a desired monoclonal antibody may be prepared using the standard hybridoma technology of Kohler and Milstein or modifications that immortalize producing B cells, as is generally known. The immortalized cell lines secreting the desired antibodies are screened by immunoassay in which the antigen is the 30P3C8 protein or a 30P3C8 fragment. When the appropriate immortalized cell culture secreting the desired antibody is identified, the cells may be expanded and antibodies produced either from in vitro cultures or from ascites fluid.

The antibodies or fragments may also be produced, using current technology, by recombinant means. Regions that bind specifically to the desired regions of the 30P3C8 protein can also be produced in the context of chimeric or CDR grafted antibodies of multiple species origin. Humanized or human 30P3C8 antibodies may also be produced and are preferred for use in therapeutic contexts. Methods for humanizing murine and other non-human antibodies by substituting one or more of the non-human antibody CDRs for corresponding human antibody sequences are well known (see for example, Jones et al., 1986, Nature 321:522-525; Riechmann et al., 1988, Nature 332:323-327; Verhoeyen et al., 1988, Science 239:1534-1536). See also, Carter et al., 1993, Proc. Natl. Acad. Sci. USA 89:4285 and Sims et al., 1993, J. Immunol. 151:2296. Methods for producing fully human monoclonal antibodies include phage display and transgenic methods (for review, see Vaughan et al., 1998, Nature Biotechnology 16:535-539).

Fully human 30P3C8 monoclonal antibodies may be generated using cloning technologies employing large human Ig gene combinatorial libraries (i.e., phage display) (Griffiths and Hoogenboom, Building an in vitro immune system: human antibodies from phage display libraries. In: Clark, M., ed., 1993, Protein Engineering of Antibody Molecules for Prophylactic and Therapeutic Applications in Man, Nottingham Academic, pp 45-64; Burton and Barbas, Human Antibodies from combinatorial libraries. Id., pp 65-82). Fully human 30P3C8 monoclonal antibodies may also be produced using transgenic mice engineered to contain human immunoglobulin gene loci as described in PCT Patent Application WO98/24893, Kucherlapati and Jakobovits et al., published Dec. 3, 1997 (see also, Jakobovits, 1998, Exp. Opin. Invest. Drugs 7(4):607-614). This method avoids the in vitro manipulation required with phage display technology and efficiently produces high affinity authentic human antibodies.

Reactivity of 30P3C8 antibodies with a 30P3C8 protein may be established by a number of well known means, including western blot, immunoprecipitation, ELISA, and FACS analyses using, as appropriate, 30P3C8 proteins, peptides, 30P3C8-expressing cells or extracts thereof.

A 30P3C8 antibody or fragment thereof of the invention may be labeled with a detectable marker or conjugated to a second molecule. Suitable detectable markers include, but are not limited to, a radioisotope, a fluorescent compound, a bioluminescent compound, chemiluminescent compound, a metal chelator or an enzyme. A second molecule for conjugation to the 30P3C8 antibody can be selected in accordance with the intended use. For example, for therapeutic use, the second molecule can be a toxin or therapeutic agent. Further, bispecific antibodies specific for two or more 30P3C8 epitopes may be generated using methods generally known in the art. Homodimeric antibodies may also be generated by cross-linking techniques known in the art (e.g., Wolff et al., 1993, Cancer Res. 53: 2560-2565).

30P3C8 Transgenic Animals

Nucleic acids that encode 30P3C8 or its modified forms can also be used to generate either transgenic animals or “knock out” animals which, in turn, are useful in the development and screening of therapeutically useful reagents. A transgenic animal (e.g., a mouse or rat) is an animal having cells that contain a transgene, which transgene was introduced into the animal or an ancestor of the animal at a prenatal, e.g., an embryonic stage. A transgene is a DNA that is integrated into the genome of a cell from which a transgenic animal develops. In one embodiment, cDNA encoding 30P3C8 can be used to clone genomic DNA encoding 30P3C8 in accordance with established techniques and the genomic sequences used to generate transgenic animals that contain cells that express DNA encoding 30P3C8. Methods for generating transgenic animals, particularly animals such as mice or rats, have become conventional in the art and are described, for example, in U.S. Pat. Nos. 4,736,866 and 4,870,009. Typically, particular cells would be targeted for 30P3C8 transgene incorporation with tissue-specific enhancers. Transgenic animals that include a copy of a transgene encoding 30P3C8 introduced into the germ line of the animal at an embryonic stage can be used to examine the effect of increased expression of DNA encoding 30P3C8. Such animals can be used as tester animals for reagents thought to confer protection from, for example, pathological conditions associated with its overexpression. In accordance with this facet of the invention, an animal is treated with the reagent and a reduced incidence of the pathological condition, compared to untreated animals bearing the transgene, would indicate a potential therapeutic intervention for the pathological condition.

Alternatively, non-human homologues of 30P3C8 can be used to construct a 30P3C8 “knock out” animal that has a defective or altered gene encoding 30P3C8 as a result of homologous recombination between the endogenous gene encoding 30P3C8 and altered genomic DNA encoding 30P3C8 introduced into an embryonic cell of the animal. For example, cDNA encoding 30P3C8 can be used to clone genomic DNA encoding 30P3C8 in accordance with established techniques. A portion of the genomic DNA encoding 30P3C8 can be deleted or replaced with another gene, such as a gene encoding a selectable marker that can be used to monitor integration. Typically, several kilobases of unaltered flanking DNA (both at the 5′ and 3′ ends) are included in the vector (see e.g., Thomas and Capecchi, 1987, Cell 51:503) for a description of homologous recombination vectors]. The vector is introduced into an embryonic stem cell line (e.g., by electroporation) and cells in which the introduced DNA has homologously recombined with the endogenous DNA are selected (see e.g., Li et al., 1992, Cell 69:915). The selected cells are then injected into a blastocyst of an animal (e.g., a mouse or rat) to form aggregation chimeras (see e.g., Bradley, in Robertson, ed., 1987, Teratocarcinomas and Embryonic Stem Cells: A Practical Approach, (IRL, Oxford), pp. 113-152). A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the 30P3C8 polypeptide.

Methods for the Detection of 30P3C8

Another aspect of the present invention relates to methods for detecting 30P3C8 polynucleotides and 30P3C8 proteins and variants thereof, as well as methods for identifying a cell that expresses 30P3C8. Northern blot analysis suggests that 30P3C8 is up-regulated in cancer, such as prostate cancer as well as other cancers. The expression profile of 30P3C8 makes it a potential diagnostic marker for local and/or metastasized disease. The status of 30P3C8 gene products may provide information useful for predicting a variety of factors including susceptibility to advanced stage disease, rate of progression, and/or tumor aggressiveness. As discussed in detail below, the status of 30P3C8 gene products in patient samples may be analyzed by a variety protocols that are well known in the art including immunohistochemical analysis, the variety of northern blotting techniques including in situ hybridization, RT-PCR analysis (for example on laser capture micro-dissected samples), western blot analysis and tissue array analysis.

More particularly, the invention provides assays for the detection of 30P3C8 polynucleotides in a biological sample, such as serum, bone, prostate, and other tissues, urine, semen, cell preparations, and the like. Detectable 30P3C8 polynucleotides include, for example, a 30P3C8 gene or fragments thereof, 30P3C8 mRNA, alternative splice variant 30P3C8 mRNAs, and recombinant DNA or RNA molecules containing a 30P3C8 polynucleotide. A number of methods for amplifying and/or detecting the presence of 30P3C8 polynucleotides are well known in the art and may be employed in the practice of this aspect of the invention.

In one embodiment, a method for detecting a 30P3C8 mRNA in a biological sample comprises producing cDNA from the sample by reverse transcription using at least one primer; amplifying the cDNA so produced using a 30P3C8 polynucleotides as sense and antisense primers to amplify 30P3C8 cDNAs therein; and detecting the presence of the amplified 30P3C8 cDNA. Optionally, the sequence of the amplified 30P3C8 cDNA can be determined. In another embodiment, a method of detecting a 30P3C8 gene in a biological sample comprises first isolating genomic DNA from the sample; amplifying the isolated genomic DNA using 30P3C8 polynucleotides as sense and antisense primers to amplify the 30P3C8 gene therein; and detecting the presence of the amplified 30P3C8 gene. Any number of appropriate sense and antisense probe combinations may be designed from the nucleotide sequences provided for the 30P3C8 (FIGS. 1A-1D (SEQ ID NO: 1)) and used for this purpose.

The invention also provides assays for detecting the presence of a 30P3C8 protein in a tissue of other biological sample such as serum, bone, prostate, and other tissues, urine, cell preparations, and the like. Methods for detecting a 30P3C8 protein are also well known and include, for example, immunoprecipitation, immunohistochemical analysis, Western Blot analysis, molecular binding assays, ELISA, ELIFA and the like. For example, in one embodiment, a method of detecting the presence of a 30P3C8 protein in a biological sample comprises first contacting the sample with a 30P3C8 antibody, a 30P3C8-reactive fragment thereof, or a recombinant protein containing an antigen binding region of a 30P3C8 antibody; and then detecting the binding of 30P3C8 protein in the sample thereto.

Methods for identifying a cell that expresses 30P3C8 are also provided. In one embodiment, an assay for identifying a cell that expresses a 30P3C8 gene comprises detecting the presence of 30P3C8 mRNA in the cell. Methods for the detection of particular mRNAs in cells are well known and include, for example, hybridization assays using complementary DNA probes (such as in situ hybridization using labeled 30P3C8 riboprobes, Northern blot and related techniques) and various nucleic acid amplification assays (such as RT-PCR using complementary primers specific for 30P3C8, and other amplification type detection methods, such as, for example, branched DNA, SISBA, TMA and the like). Alternatively, an assay for identifying a cell that expresses a 30P3C8 gene comprises detecting the presence of 30P3C8 protein in the cell or secreted by the cell. Various methods for the detection of proteins are well known in the art and may be employed for the detection of 30P3C8 proteins and 30P3C8 expressing cells.

30P3C8 expression analysis may also be useful as a tool for identifying and evaluating agents that modulate 30P3C8 gene expression. For example, 30P3C8 expression is significantly upregulated in prostate cancer, and may also be expressed in other cancers. Identification of a molecule or biological agent that could inhibit 30P3C8 expression or over-expression in cancer cells may be of therapeutic value. Such an agent may be identified by using a screen that quantifies 30P3C8 expression by RT-PCR, nucleic acid hybridization or antibody binding.

Monitoring the Status of 30P3C8 and its Products

Assays that evaluate the status of the 30P3C8 gene and 30P3C8 gene products in an individual may provide information on the growth or oncogenic potential of a biological sample from this individual. For example, because 30P3C8 mRNA is so highly expressed in prostate cancer lines as compared to normal prostate tissue, assays that evaluate the relative levels of 30P3C8 mRNA transcripts or proteins in a biological sample may be used to diagnose a disease associated with 30P3C8 disregulation such as cancer and may provide prognostic information useful in defining appropriate therapeutic options. Similarly, assays that evaluate the integrity 30P3C8 nucleotide and amino acid sequences in a biological sample, may also be used in this context.

The finding that 30P3C8 mRNA is so highly expressed in prostate cancer lines as compared to normal prostate tissue provides evidence that this gene is associated with disregulated cell growth and therefore identifies this gene and its products as targets that the skilled artisan can use to evaluate biological samples from individuals suspected of having a disease associated with 30P3C8 disregulation. In this context, the evaluation of the expression status of 30P3C8 gene and its products can be used to gain information on the disease potential of a tissue sample. The terms “expression status” in this context is used to broadly refer to the variety of factors involved in the expression, function and regulation of a gene and its products such as the level of mRNA expression, the integrity of the expressed gene products (such as the nucleic and amino acid sequences) and transcriptional and translational modifications to these molecules.

The expression status of 30P3C8 may provide information useful for predicting susceptibility to particular disease stages, progression, and/or tumor aggressiveness. The invention provides methods and assays for determining 30P3C8 expression status and diagnosing cancers that express 30P3C8, such as cancers of the 30P3C8 expression status in patient samples may be analyzed by a number of means well known in the art, including without limitation, immunohistochemical analysis, in situ hybridization, RT-PCR analysis on laser capture micro-dissected samples, western blot analysis of clinical samples and cell lines, and tissue array analysis. Typical protocols for evaluating the expression status of the 30P3C8 gene and gene products can be found, for example in Ausubul et al. eds., 1995, Current Protocols In Molecular Biology, Units 2 [Northern Blotting], 4 [Southern Blotting], 15 [Immunoblotting] and 18 [PCR Analysis].

In one aspect, the invention provides methods for monitoring 30P3C8 gene products by determining the status of 30P3C8 gene products expressed by cells in a test tissue sample from an individual suspected of having a disease associated with disregulated cell growth (such as hyperplasia or cancer) and then comparing the status so determined to the status of 30P3C8 gene products in a corresponding normal sample, the presence of aberrant 30P3C8 gene products in the test sample relative to the normal sample providing an indication of the presence of disregulated cell growth within the cells of the individual.

In another aspect, the invention provides assays useful in determining the presence of cancer in an individual, comprising detecting a significant increase in 30P3C8 mRNA or protein expression in a test cell or tissue sample relative to expression levels in the corresponding normal cell or tissue. The presence of 30P3C8 mRNA may, for example, be evaluated in tissue samples including but not limited to colon, lung, prostate, pancreas, bladder, breast, ovary, cervix, testis, head and neck, brain, stomach, bone, etc. The presence of significant 30P3C8 expression in any of these tissues may be useful to indicate the emergence, presence and/or severity of these cancers, since the corresponding normal tissues do not express 30P3C8 mRNA or express it at lower levels.

In a related embodiment, 30P3C8 expression status may be determined at the protein level rather than at the nucleic acid level. For example, such a method or assay would comprise determining the level of 30P3C8 protein expressed by cells in a test tissue sample and comparing the level so determined to the level of 30P3C8 expressed in a corresponding normal sample. In one embodiment, the presence of 30P3C8 protein is evaluated, for example, using immunohistochemical methods. 30P3C8 antibodies or binding partners capable of detecting 30P3C8 protein expression may be used in a variety of assay formats well known in the art for this purpose.

In other related embodiments, one can evaluate the integrity 30P3C8 nucleotide and amino acid sequences in a biological sample in order to identify perturbations in the structure of these molecules such as insertions, deletions, substitutions and the like. Such embodiments are useful because perturbations in the nucleotide and amino acid sequences are observed in a large number of proteins associated with a growth disregulated phenotype (see, e.g., Marrogi et al., 1999, J. Cutan. Pathol. 26(8):369-378). In this context, a wide variety of assays for observing perturbations in nucleotide and amino acid sequences are well known in the art. For example, the size and structure of nucleic acid or amino acid sequences of 30P3C8 gene products may be observed by the Northern, Southern, Western, PCR and DNA sequencing protocols discussed herein. In addition, other methods for observing perturbations in nucleotide and amino acid sequences such as single strand conformation polymorphism analysis are well known in the art (see, e.g., U.S. Pat. Nos. 5,382,510 and 5,952,170).

In another related embodiment, the invention provides assays useful in determining the presence of cancer in an individual, comprising detecting a significant change in the 30P3C8 alternative splice variants expressed in a test cell or tissue sample relative to expression levels in the corresponding normal cell or tissue. The monitoring of alternative splice variants of 30P3C8 is useful because changes in the alternative splicing of proteins is suggested as one of the steps in a series of events that lead to the progression of cancers (see e.g. Carstens et al., 1997, Oncogene 15(25):3059-3065). Moreover, the differential expression of the 30P3C8 transcripts in cancer tissue cell lines (FIGS. 4-6) provides evidence that alternative splicing of 30P3C8 plays a role in the malignant phenotype.

In addition to the tissues discussed above, peripheral blood may be conveniently assayed for the presence of cancer cells, including but not limited to prostate, pancreatic, colon, brain, bone, lung, kidney and bladder cancers, using for example, northern or RT-PCR analysis to detect 30P3C8 expression (see e.g. FIG. 5). The presence of RT-PCR amplifiable 30P3C8 mRNA provides an indication of the presence of the cancer. RT-PCR detection assays for tumor cells in peripheral blood are currently being evaluated for use in the diagnosis and management of a number of human solid tumors. In the prostate cancer field, these include RT-PCR assays for the detection of cells expressing PSA and PSM (Verkaik et al., 1997, Urol. Res. 25:373-384; Ghossein et al., 1995, J. Clin. Oncol. 13:1195-2000; Heston et al., 1995, Clin. Chem. 41:1687-1688). RT-PCR assays are well known in the art.

A related aspect of the invention is directed to predicting susceptibility to developing cancer in an individual. In one embodiment, a method for predicting susceptibility to cancer comprises detecting 30P3C8 mRNA or 30P3C8 protein in a tissue sample, its presence indicating susceptibility to cancer, wherein the degree of 30P3C8 mRNA expression present is proportional to the degree of susceptibility. In a specific embodiment, the presence of 30P3C8 in prostate tissue is examined, with the presence of 30P3C8 in the sample providing an indication of prostate cancer susceptibility (or the emergence or existence of a prostate tumor). In another specific embodiment, the presence of 30P3C8 in pancreatic, colon, brain, bone, lung, kidney or bladder tissue is examined, with the presence of 30P3C8 in the sample providing an indication of cancer susceptibility (or the emergence or existence of a tumor). In a closely related embodiment, one can evaluate the integrity 30P3C8 nucleotide and amino acid sequences in a biological sample in order to identify perturbations in the structure of these molecules such as insertions, deletions, substitutions and the like, with the presence of one or more perturbations in 30P3C8 gene products in the sample providing an indication of cancer susceptibility (or the emergence or existence of a tumor).

Yet another related aspect of the invention is directed to methods for gauging tumor aggressiveness. In one embodiment, a method for gauging aggressiveness of a tumor comprises determining the level of 30P3C8 mRNA or 30P3C8 protein expressed by cells in a sample of the tumor, comparing the level so determined to the level of 30P3C8 mRNA or 30P3C8 protein expressed in a corresponding normal tissue taken from the same individual or a normal tissue reference sample, wherein the degree of 30P3C8 mRNA or 30P3C8 protein expression in the tumor sample relative to the normal sample indicates the degree of aggressiveness. In a specific embodiment, aggressiveness of prostate, pancreatic, colon, brain, bone, lung, kidney or bladder tumors is evaluated by determining the extent to which 30P3C8 is expressed in the tumor cells, with higher expression levels indicating more aggressive tumors. In a closely related embodiment, one can evaluate the integrity 30P3C8 nucleotide and amino acid sequences in a biological sample in order to identify perturbations in the structure of these molecules such as insertions, deletions, substitutions and the like, with the presence of one or more perturbations indicating more aggressive tumors.

Yet another related aspect of the invention is directed to methods for observing the progression of a malignancy in an individual over time. In one embodiment, methods for observing the progression of a malignancy in an individual over time comprise determining the level of 30P3C8 mRNA or 30P3C8 protein expressed by cells in a sample of the tumor, comparing the level so determined to the level of 30P3C8 mRNA or 30P3C8 protein expressed in an equivalent tissue sample taken from the same individual at a different time, wherein the degree of 30P3C8 mRNA or 30P3C8 protein expression in the tumor sample over time provides information on the progression of the cancer. In a specific embodiment, the progression of a cancer is evaluated by determining the extent to which 30P3C8 expression in the tumor cells alters over time, with higher expression levels indicating a progression of the cancer. In a closely related embodiment, one can evaluate the integrity 30P3C8 nucleotide and amino acid sequences in a biological sample in order to identify perturbations in the structure of these molecules such as insertions, deletions, substitutions and the like, with the presence of one or more perturbations indicating a progression of the cancer.

The above diagnostic approaches may be combined with any one of a wide variety of prognostic and diagnostic protocols known in the art. For example, another embodiment of the invention disclosed herein is directed to methods for observing a coincidence between the expression of 30P3C8 gene and 30P3C8 gene products (or perturbations in 30P3C8 gene and 30P3C8 gene products) and a factor that is associated with malignancy as a means of diagnosing and prognosticating the status of a tissue sample. In this context, a wide variety of factors associated with malignancy may be utilized such as the expression of genes otherwise associated with malignancy (including PSA, PSCA and PSM expression) as well as gross cytological observations (see e.g. Bocking et al., 1984, Anal. Quant. Cytol. 6(2):74-88; Eptsein, 1995, Hum. Pathol. 26(2):223-9; Thorson et al., 1998, Mod. Pathol. 11(6):543-51; Baisden et al., 1999, Am. J. Surg. Pathol. 23(8):918-24). Methods for observing a coincidence between the expression of 30P3C8 gene and 30P3C8 gene products (or perturbations in 30P3C8 gene and 30P3C8 gene products) and an additional factor that is associated with malignancy are useful, for example, because the presence of a set or constellation of specific factors that coincide provides information crucial for diagnosing and prognosticating the status of a tissue sample.

In a typical embodiment, methods for observing a coincidence between the expression of 30P3C8 gene and 30P3C8 gene products (or perturbations in 30P3C8 gene and 30P3C8 gene products) and a factor that is associated with malignancy entails detecting the overexpression of 30P3C8 mRNA or protein in a tissue sample, detecting the overexpression of PSA mRNA or protein in a tissue sample, and observing a coincidence of 30P3C8 mRNA or protein and PSA mRNA or protein overexpression. In a specific embodiment, the expression of 30P3C8 and PSA mRNA in prostate tissue is examined. In a preferred embodiment, the coincidence of 30P3C8 and PSA mRNA overexpression in the sample provides an indication of prostate cancer, prostate cancer susceptibility or the emergence or existence of a prostate tumor.

Methods for detecting and quantifying the expression of 30P3C8 mRNA or protein are described herein and use of standard nucleic acid and protein detection and quantification technologies is well known in the art. Standard methods for the detection and quantification of 30P3C8 mRNA include in situ hybridization using labeled 30P3C8 riboprobes, northern blot and related techniques using 30P3C8 polynucleotide probes, RT-PCR analysis using primers specific for 30P3C8, and other amplification type detection methods, such as, for example, branched DNA, SISBA, TMA and the like. In a specific embodiment, semi-quantitative RT-PCR may be used to detect and quantify 30P3C8 mRNA expression as described in the Examples that follow. Any number of primers capable of amplifying 30P3C8 may be used for this purpose, including but not limited to the various primer sets specifically described herein. Standard methods for the detection and quantification of protein may be used for this purpose. In a specific embodiment, polyclonal or monoclonal antibodies specifically reactive with the wild-type 30P3C8 protein may be used in an immunohistochemical assay of biopsied tissue.

Identifying Molecules that Interact with 30P3C8

The 30P3C8 protein sequences disclosed herein allow the skilled artisan to identify molecules that interact with them via any one of a variety of art accepted protocols. For example one can utilize one of the variety of so-called interaction trap systems (also referred to as the “two-hybrid assay”). In such systems, molecules that interact reconstitute a transcription factor and direct expression of a reporter gene, the expression of which is then assayed. Typical systems identify protein-protein interactions in vivo through reconstitution of a eukaryotic transcriptional activator and are disclosed for example in U.S. Pat. Nos. 5,955,280, 5,925,523, 5,846,722 and 6,004,746.

Alternatively one can identify molecules that interact with 30P3C8 protein sequences by screening peptide libraries. In such methods, peptides that bind to selected receptor molecules such as 30P3C8 are identified by screening libraries that encode a random or controlled collection of amino acids. Peptides encoded by the libraries are expressed as fusion proteins of bacteriophage coat proteins, and bacteriophage particles are then screened against the receptors of interest. Peptides having a wide variety of uses, such as therapeutic or diagnostic reagents, may thus be identified without any prior information on the structure of the expected ligand or receptor molecule. Typical peptide libraries and screening methods that can be used to identify molecules that interact with 30P3C8 protein sequences are disclosed for example in U.S. Pat. Nos. 5,723,286 and 5,733,731.

Alternatively, cell lines expressing 30P3C8 can be used to identify protein-protein interactions mediated by 30P3C8. This possibility can be examined using immunoprecipitation techniques as shown by others (Hamilton, B. J., et al., 1999, Biochem. Biophys. Res. Commun. 261:646-51). Typically 30P3C8 protein can be immunoprecipitated from 30P3C8 expressing prostate cancer cell lines using anti-30P3C8 antibodies. Alternatively, antibodies against His-tag can be used in cell line engineered to express 30P3C8 (vectors mentioned above). The immunoprecipitated complex can be examined for protein association by procedures such as western blotting, ³⁵S-methionine labeling of proteins, protein microsequencing, silver staining and two dimensional gel electrophoresis.

Related embodiments of such screening assays include methods for identifying small molecules that interact with 30P3C8. Typical methods are discussed for example in U.S. Pat. No.5,928,868 and include methods for forming hybrid ligands in which at least one ligand is a small molecule. In an illustrative embodiments, the hybrid ligand is introduced into cells that in turn contain a first and a second expression vector. Each expression vector includes DNA for expressing a hybrid protein that encodes a target protein linked to a coding sequence for a transcriptional module. The cells further contains a reporter gene, the expression of which is conditioned on the proximity of the first and second hybrid proteins to each other, an event that occurs only if the hybrid ligand binds to target sites on both hybrid proteins. Those cells that express the reporter gene are selected and the unknown small molecule or the unknown hybrid protein is identified.

A typical embodiment of this invention consists of a method of screening for a molecule that interacts with a 30P3C8 amino acid sequence shown in FIGS. 1A-1D (SEQ ID NO: 2), comprising the steps of contacting a population of molecules with the 30P3C8 amino acid sequence, allowing the population of molecules and the 30P3C8 amino acid sequence to interact under conditions that facilitate an interaction, determining the presence of a molecule that interacts with the 30P3C8 amino acid sequence and then separating molecules that do not interact with the 30P3C8 amino acid sequence from molecules that do interact with the 30P3C8 amino acid sequence. In a specific embodiment, the method further includes purifying a molecule that interacts with the 30P3C8 amino acid sequence. In a preferred embodiment, the 30P3C8 amino acid sequence is contacted with a library of peptides.

Therapeutic Methods and Compositions

The identification of 30P3C8 as a gene that is highly expressed in cancers of the prostate (and other cancers), opens a number of therapeutic approaches to the treatment of such cancers. Accordingly, therapeutic approaches aimed at inhibiting the activity of the 30P3C8 protein are expected to be useful for patients suffering from prostate cancer, and other cancers expressing 30P3C8. These therapeutic approaches aimed at inhibiting the activity of the 30P3C8 protein generally fall into two classes. One class comprises various methods for inhibiting the binding or association of the 30P3C8 protein with its binding partner or with other proteins. Another class comprises a variety of methods for inhibiting the transcription of the 30P3C8 gene or translation of 30P3C8 mRNA.

30P3C8 antibodies can be introduced into a patient such that the antibody binds to 30P3C8 in serum or blood, for example, where it can modulate binding to a receptor or other binding partner or growth factor, thereby inhibiting the growth or metastasis of cells or a tumor. 30P3C8 antibodies can be conjugated to toxic or therapeutic agents and used to deliver the toxic or therapeutic agent directly to 30P3C8-associated tumor cells. Examples of toxic agents include, but are not limited to, calchemicin, maytansinoids, radioisotopes such as ¹³¹I, ytrium, and bismuth.

Cancer immunotherapy using anti-30P3C8 antibodies may follow the teachings generated from various approaches that have been successfully employed in the treatment of other types of cancer, including but not limited to colon cancer (Arlen et al., 1998, Crit. Rev. Immunol. 18:133-138), multiple myeloma (Ozaki et al., 1997, Blood 90:3179-3186; Tsunenari et al., 1997, Blood 90:2437-2444), gastric cancer (asprzyk et al., 1992, Cancer Res. 52:2771-2776), B-cell lymphoma (Funakoshi et al., 1996, J. Immunother. Emphasis Tumor Immunol. 19:93-101), leukemia (Zhong et al., 1996, Leuk. Res. 20:581-589), colorectal cancer (Moun et al., 1994, Cancer Res. 54:6160-6166; Velders et al., 1995, Cancer Res. 55:4398-4403), and breast cancer (Shepard et al., 1991, J. Clin. Immunol. 11:117-127). Some therapeutic approaches involve conjugation of naked antibody to a toxin, such as the conjugation of ¹³¹I to anti-CD20 antibodies (Coulter Pharmaceuticals, Palo Alto, Calif.), while others involve co-administration of antibodies and other therapeutic agents, such as Herceptin™ (trastuzumab) with paclitaxel (Genentech, Inc.). For treatment of prostate cancer, for example, 30P3C8 antibodies can be administered in conjunction with radiation, chemotherapy or hormone ablation.

Although 30P3C8 antibody therapy may be useful for all stages of cancer, antibody therapy may be particularly appropriate in advanced or metastatic cancers. Treatment with the antibody therapy of the invention may be indicated for patients who have received previously one or more chemotherapy, while combining the antibody therapy of the invention with a chemotherapeutic or radiation regimen may be preferred for patients who have not received chemotherapeutic treatment. Additionally, antibody therapy may enable the use of reduced dosages of concomitant chemotherapy, particularly for patients who do not tolerate the toxicity of the chemotherapeutic agent very well.

It may be desirable for some cancer patients to be evaluated for the presence and level of 30P3C8 expression, preferably using immunohistochemical assessments of tumor tissue, quantitative 30P3C8 imaging, or other techniques capable of reliably indicating the presence and degree of 30P3C8 expression. Immunohistochemical analysis of tumor biopsies or surgical specimens may be preferred for this purpose. Methods for immunohistochemical analysis of tumor tissues are well known in the art.

Anti-30P3C8 monoclonal antibodies useful in treating prostate and other cancers include those that are capable of initiating a potent immune response against the tumor and those that are capable of interfering with binding of 30P3C8 with receptors or other binding partners. In this regard, anti-30P3C8 antibodies may bind to 30P3C8 and disrupt interactions between 30P3C8 and other proteins, such as receptors for which 30P3C8 is a ligand. Because 30P3C8 may be a growth factor or similar molecule involved in tumor growth and metastasis, anti-30P3C8 antibodies may inhibit tumor growth and/or metastasis by disrupting the homing or invasion or other cancer-promoting activities of 30P3C8. In addition, anti-30P3C8 mAbs that exert a direct biological effect on tumor growth are useful in the practice of the invention.

The use of murine or other non-human monoclonal antibodies, or human/mouse chimeric mAbs may induce moderate to strong immune responses in some patients. In some cases, this will result in clearance of the antibody from circulation and reduced efficacy. In the most severe cases, such an immune response may lead to the extensive formation of immune complexes which, potentially, can cause renal failure. Accordingly, preferred monoclonal antibodies used in the practice of the therapeutic methods of the invention are those that are either fully human or humanized and that bind specifically to the target 30P3C8 antigen with high affinity but exhibit low or no antigenicity in the patient.

Therapeutic methods of the invention contemplate the administration of single anti-30P3C8 mAbs as well as combinations, or cocktails, of different mAbs. Such mAb cocktails may have certain advantages inasmuch as they contain mAbs that target different epitopes, exploit different effector mechanisms or combine directly cytotoxic mAbs with mAbs that rely on immune effector functionality. Such mAbs in combination may exhibit synergistic therapeutic effects. In addition, the administration of anti-30P3C8 mAbs may be combined with other therapeutic agents, including but not limited to various chemotherapeutic agents, androgen-blockers, and immune modulators (e.g., IL-2, GM-CSF). The anti-30P3C8 mAbs may be administered in their “naked” or unconjugated form, or may have therapeutic agents conjugated to them.

The anti-30P3C8 antibody formulations may be administered via any route capable of delivering the antibodies to the tumor site. Potentially effective routes of administration include, but are not limited to, intravenous, intraperitoneal, intramuscular, intratumor, intradermal, and the like. Treatment will generally involve the repeated administration of the anti-30P3C8 antibody preparation via an acceptable route of administration such as intravenous injection (IV), typically at a dose in the range of about 0.1 to about 10 mg/kg body weight. Doses in the range of 10-500 mg mAb per week may be effective and well tolerated.

Based on clinical experience with the Herceptin mAb in the treatment of metastatic breast cancer, an initial loading dose of approximately 4 mg/kg patient body weight IV followed by weekly doses of about 2 mg/kg IV of the anti-30P3C8 mAb preparation may represent an acceptable dosing regimen. Preferably, the initial loading dose is administered as a 90 minute or longer infusion. The periodic maintenance dose may be administered as a 30 minute or longer infusion, provided the initial dose was well tolerated. However, as one of skill in the art will understand, various factors will influence the ideal dose regimen in a particular case. Such factors may include, for example, the binding affinity and half life of the Ab or mAbs used, the degree of 30P3C8 expression in the patient, the extent of circulating shed 30P3C8 antigen, the desired steady-state antibody concentration level, frequency of treatment, and the influence of chemotherapeutic agents used in combination with the treatment method of the invention.

Optimally, patients should be evaluated for the level of circulating shed 30P3C8 antigen in serum in order to assist in the determination of the most effective dosing regimen and related factors. Such evaluations may also be used for monitoring purposes throughout therapy, and may be useful to gauge therapeutic success in combination with evaluating other parameters (such as serum PSA levels in prostate cancer therapy).

Inhibition of 30P3C8 Protein Function

The invention includes various methods and compositions for inhibiting the binding of 30P3C8 to its binding partner or ligand, or its association with other protein(s) as well as methods for inhibiting 30P3C8 function.

Inhibition of 30P3C8 with Intracellular Antibodies

In one approach, recombinant vectors encoding single chain antibodies that specifically bind to 30P3C8 may be introduced into 30P3C8 expressing cells via gene transfer technologies, wherein the encoded single chain anti-30P3C8 antibody is expressed intracellularly, binds to 30P3C8 protein, and thereby inhibits its function. Methods for engineering such intracellular single chain antibodies are well known. Such intracellular antibodies, also known as “intrabodies”, may be specifically targeted to a particular compartment within the cell, providing control over where the inhibitory activity of the treatment will be focused. This technology has been successfully applied in the art (for review, see Richardson and Marasco, 1995, TIBTECH vol. 13). Intrabodies have been shown to virtually eliminate the expression of otherwise abundant cell surface receptors. See, for example, Richardson et al., 1995, Proc. Natl. Acad. Sci. USA 92: 3137-3141; Beerli et al., 1994, J. Biol. Chem. 289: 23931-23936; Deshane et al., 1994, Gene Ther. 1: 332-337.

Single chain antibodies comprise the variable domains of the heavy and light chain joined by a flexible linker polypeptide, and are expressed as a single polypeptide. Optionally, single chain antibodies may be expressed as a single chain variable region fragment joined to the light chain constant region. Well known intracellular trafficking signals may be engineered into recombinant polynucleotide vectors encoding such single chain antibodies in order to precisely target the expressed intrabody to the desired intracellular compartment. For example, intrabodies targeted to the endoplasmic reticulum (ER) may be engineered to incorporate a leader peptide and, optionally, a C-terminal ER retention signal, such as the KDEL amino acid motif. Intrabodies intended to exert activity in the nucleus may be engineered to include a nuclear localization signal. Lipid moieties may be joined to intrabodies in order to tether the intrabody to the cytosolic side of the plasma membrane. Intrabodies may also be targeted to exert function in the cytosol. For example, cytosolic intrabodies may be used to sequester factors within the cytosol, thereby preventing them from being transported to their natural cellular destination.

In one embodiment, intrabodies may be used to capture 30P3C8 in the nucleus, thereby preventing its activity within the nucleus. Nuclear targeting signals may be engineered into such 30P3C8 intrabodies in order to achieve the desired targeting. Such 30P3C8 intrabodies may be designed to bind specifically to a particular 30P3C8 domain. In another embodiment, cytosolic intrabodies that specifically bind to the 30P3C8 protein may be used to prevent 30P3C8 from gaining access to the nucleus, thereby preventing it from exerting any biological activity within the nucleus (e.g., preventing 30P3C8 from forming transcription complexes with other factors).

In order to specifically direct the expression of such intrabodies to particular tumor cells, the transcription of the intrabody may be placed under the regulatory control of an appropriate tumor-specific promoter and/or enhancer. In order to target intrabody expression specifically to prostate, for example, the PSA promoter and/or promoter/enhancer may be utilized (See, for example, U.S. Pat. No. 5,919,652).

Inhibition of 30P3C8 with Recombinant Proteins

In another approach, recombinant molecules that are capable of binding to 30P3C8 thereby preventing 30P3C8 from accessing/binding to its binding partner(s) or associating with other protein(s) are used to inhibit 30P3C8 function. Such recombinant molecules may, for example, contain the reactive part(s) of a 30P3C8 specific antibody molecule. In a particular embodiment, the 30P3C8 binding domain of a 30P3C8 binding partner may be engineered into a dimeric fusion protein comprising two 30P3C8 ligand binding domains linked to the Fc portion of a human IgG, such as human IgG1. Such IgG portion may contain, for example, the C_{H}2 and C_{H}3 domains and the hinge region, but not the C_{H}1 domain. Such dimeric fusion proteins may be administered in soluble form to patients suffering from a cancer associated with the expression of 30P3C8, including but not limited to prostate, pancreatic, colon, brain, bone, lung, kidney and bladder cancers, where the dimeric fusion protein specifically binds to 30P3C8 thereby blocking 30P3C8 interaction with a binding partner. Such dimeric fusion proteins may be further combined into multimeric proteins using known antibody linking technologies.

Inhibition of 30P3C8 Transcription or Translation

Within another class of therapeutic approaches, the invention provides various methods and compositions for inhibiting the transcription of the 30P3C8 gene. Similarly, the invention also provides methods and compositions for inhibiting the translation of 30P3C8 mRNA into protein.

In one approach, a method of inhibiting the transcription of the 30P3C8 gene comprises contacting the 30P3C8 gene with a 30P3C8 antisense polynucleotide. In another approach, a method of inhibiting 30P3C8 mRNA translation comprises contacting the 30P3C8 mRNA with an antisense polynucleotide. In another approach, a 30P3C8 specific ribozyme may be used to cleave the 30P3C8 message, thereby inhibiting translation. Such antisense and ribozyme based methods may also be directed to the regulatory regions of the 30P3C8 gene, such as the 30P3C8 promoter and/or enhancer elements. Similarly, proteins capable of inhibiting a 30P3C8 gene transcription factor may be used to inhibit 30P3C8 mRNA transcription. The various polynucleotides and compositions useful in the aforementioned methods have been described above. The use of antisense and ribozyme molecules to inhibit transcription and translation is well known in the art.

Other factors that inhibit the transcription of 30P3C8 through interfering with 30P3C8 transcriptional activation may also be useful for the treatment of cancers expressing 30P3C8. Similarly, factors that are capable of interfering with 30P3C8 processing may be useful for the treatment of cancers expressing 30P3C8. Cancer treatment methods utilizing such factors are also within the scope of the invention.

General Considerations for Therapeutic Strategies

Gene transfer and gene therapy technologies may be used for delivering therapeutic polynucleotide molecules to tumor cells synthesizing 30P3C8 (i.e., antisense, ribozyme, polynucleotides encoding intrabodies and other 30P3C8 inhibitory molecules). A number of gene therapy approaches are known in the art. Recombinant vectors encoding 30P3C8 antisense polynucleotides, ribozymes, factors capable of interfering with 30P3C8 transcription, and so forth, may be delivered to target tumor cells using such gene therapy approaches.

The above therapeutic approaches may be combined with any one of a wide variety of chemotherapy or radiation therapy regimens. These therapeutic approaches may also enable the use of reduced dosages of chemotherapy and/or less frequent administration, particularly in patients that do not tolerate the toxicity of the chemotherapeutic agent well.

The anti-tumor activity of a particular composition (e.g., antisense, ribozyme, intrabody), or a combination of such compositions, may be evaluated using various in vitro and in vivo assay systems. In vitro assays for evaluating therapeutic potential include cell growth assays, soft agar assays and other assays indicative of tumor promoting activity, binding assays capable of determining the extent to which a therapeutic composition will inhibit the binding of 30P3C8 to a binding partner, etc.

In vivo, the effect of a 30P3C8 therapeutic composition may be evaluated in a suitable animal model. For example, xenogenic prostate cancer models wherein human prostate cancer explants or passaged xenograft tissues are introduced into immune compromised animals, such as nude or SCID mice, are appropriate in relation to prostate cancer and have been described (Klein et al., 1997, Nature Medicine 3:402-408). For example, PCT Patent Application WO98/16628, Sawyers et al., published Apr. 23, 1998, describes various xenograft models of human prostate cancer capable of recapitulating the development of primary tumors, micrometastasis, and the formation of osteoblastic metastases characteristic of late stage disease. Efficacy may be predicted using assays that measure inhibition of tumor formation, tumor regression or metastasis, and the like. See, also, the Examples below.

In vivo assays that qualify the promotion of apoptosis may also be useful in evaluating potential therapeutic compositions. In one embodiment, xenografts from bearing mice treated with the therapeutic composition may be examined for the presence of apoptotic foci and compared to untreated control xenograft-bearing mice. The extent to which apoptotic foci are found in the tumors of the treated mice provides an indication of the therapeutic efficacy of the composition.

The therapeutic compositions used in the practice of the foregoing methods may be formulated into pharmaceutical compositions comprising a carrier suitable for the desired delivery method. Suitable carriers include any material that when combined with the therapeutic composition retains the anti-tumor function of the therapeutic composition and is non-reactive with the patient's immune system. Examples include, but are not limited to, any of a number of standard pharmaceutical carriers such as sterile phosphate buffered saline solutions, bacteriostatic water, and the like (see, generally, Remington's Pharmaceutical Sciences 16^{th} Ed., A. Osal., Ed., 1980).

Therapeutic formulations may be solubilized and administered via any route capable of delivering the therapeutic composition to the tumor site. Potentially effective routes of administration include, but are not limited to, intravenous, parenteral, intraperitoneal, intramuscular, intratumor, intradermal, intraorgan, orthotopic, and the like. A preferred formulation for intravenous injection comprises the therapeutic composition in a solution of preserved bacteriostatic water, sterile unpreserved water, and/or diluted in polyvinylchloride or polyethylene bags containing 0.9% sterile sodium chloride for injection, USP. Therapeutic protein preparations may be lyophilized and stored as sterile powders, preferably under vacuum, and then reconstituted in bacteriostatic water containing, for example, benzyl alcohol preservative, or in sterile water prior to injection.

Dosages and administration protocols for the treatment of cancers using the foregoing methods will vary with the method and the target cancer and will generally depend on a number of other factors appreciated in the art.

Cancer Vaccines

The invention further provides cancer vaccines comprising a 30P3C8 protein or fragment thereof, as well as DNA based vaccines. Preferably, the vaccine comprises an immunogenic portion of a 30P3C8 protein or polypeptide. In view of the over-expression of 30P3C8 in tumors, cancer vaccines are expected to be effective at preventing and/or treating 30P3C8 expressing cancers. The use of a tumor antigen in a vaccine for generating humoral and cell-mediated immunity for use in anti-cancer therapy is well known in the art and has been employed in prostate cancer using human PSMA and rodent PAP immunogens (Hodge et al., 1995, Int. J. Cancer 63:231-237; Fong et al., 1997, J. Immunol. 159:3113-3117). Such methods can be readily practiced by employing a 30P3C8 protein, or fragment thereof, or a 30P3C8-encoding nucleic acid molecule and recombinant vectors capable of expressing and appropriately presenting the 30P3C8 immunogen.

For example, viral gene delivery systems may be used to deliver a 30P3C8-encoding nucleic acid molecule. Various viral gene delivery systems that can be used in the practice of this aspect of the invention include, but are not limited to, vaccinia, fowlpox, canarypox, adenovirus, influenza, poliovirus, adeno-associated virus, lentivirus, and sindbus virus (Restifo, 1996, Curr. Opin. Immunol. 8:658-663). Non-viral delivery systems may also be employed by using naked DNA encoding a 30P3C8 protein or fragment thereof introduced into the patient (e.g., intramuscularly) to induce an anti-tumor response. In one embodiment, the full-length human 30P3C8 cDNA may be employed. In another embodiment, 30P3C8 nucleic acid molecules encoding specific cytotoxic T lymphocyte (CTL) epitopes may be employed. CTL epitopes can be determined using specific algorithms (e.g., Epimer, Brown University) to identify peptides within a 30P3C8 protein that are capable of optimally binding to specified HLA alleles.

Various ex vivo strategies may also be employed. One approach involves the use of dendritic cells to present 30P3C8 antigen to a patient's immune system. Dendritic cells express MHC class I and II, B7 co-stimulator, and IL-12, and are thus highly specialized antigen presenting cells. In prostate cancer, autologous dendritic cells pulsed with peptides of the prostate-specific membrane antigen (PSMA) are being used in a Phase I clinical trial to stimulate prostate cancer patients' immune systems (Tjoa et al., 1996, Prostate 28:65-69; Murphy et al., 1996, Prostate 29:371-380). Dendritic cells can be used to present 30P3C8 peptides to T cells in the context of MHC class I and II molecules. In one embodiment, autologous dendritic cells are pulsed with 30P3C8 peptides capable of binding to MHC molecules. In another embodiment, dendritic cells are pulsed with the complete 30P3C8 protein. Yet another embodiment involves engineering the overexpression of the 30P3C8 gene in dendritic cells using various implementing vectors known in the art, such as adenovirus (Arthur et al., 1997, Cancer Gene Ther. 4:17-25), retrovirus (Henderson et al., 1996, Cancer Res. 56:3763-3770), lentivirus, adeno-associated virus, DNA transfection (Ribas et al., 1997, Cancer Res. 57:2865-2869), and tumor-derived RNA transfection (Ashley et al., 1997, J. Exp. Med. 186:1177-1182). Cells expressing 30P3C8 may also be engineered to express immune modulators, such as GM-CSF, and used as immunizing agents.

Anti-idiotypic anti-30P3C8 antibodies can also be used in anti-cancer therapy as a vaccine for inducing an immune response to cells expressing a 30P3C8 protein. Specifically, the generation of anti-idiotypic antibodies is well known in the art and can readily be adapted to generate anti-idiotypic anti-30P3C8 antibodies that mimic an epitope on a 30P3C8 protein (see, for example, Wagner et al., 1997, Hybridoma 16: 3340; Foon et al., 1995, J. Clin. Invest. 96:334-342; Herlyn et al., 1996, Cancer Immunol. Immunother. 43:65-76). Such an anti-idiotypic antibody can be used in cancer vaccine strategies.

Genetic immunization methods may be employed to generate prophylactic or therapeutic humoral and cellular immune responses directed against cancer cells expressing 30P3C8. Constructs comprising DNA encoding a 30P3C8 protein/immunogen and appropriate regulatory sequences may be injected directly into muscle or skin of an individual, such that the cells of the muscle or skin take-up the construct and express the encoded 30P3C8 protein/immunogen. Expression of the 30P3C8 protein immunogen results in the generation of prophylactic or therapeutic humoral and cellular immunity against prostate, pancreatic, colon, brain, bone, lung, kidney and/or bladder cancers. Various prophylactic and therapeutic genetic immunization techniques known in the art may be used (for review, see information and references published at Internet address www.genweb.com).

Kits

For use in the diagnostic and therapeutic applications described or suggested above, kits are also provided by the invention. Such kits may comprise a carrier means being compartmentalized to receive in close confinement one or more container means such as vials, tubes, and the like, each of the container means comprising one of the separate elements to be used in the method. For example, one of the container means may comprise a probe that is or can be detectably labeled. Such probe may be an antibody or polynucleotide specific for a 30P3C8 protein or a 30P3C8 gene or message, respectively. Where the kit utilizes nucleic acid hybridization to detect the target nucleic acid, the kit may also have containers containing nucleotide(s) for amplification of the target nucleic acid sequence and/or a container comprising a reporter-means, such as a biotin-binding protein, such as avidin or streptavidin, bound to a reporter molecule, such as an enzymatic, florescent, or radioisotope label.

The kit of the invention will typically comprise the container described above and one or more other containers comprising materials desirable from a commercial and user standpoint, including buffers, diluents, filters, needles, syringes, and package inserts with instructions for use. A label may be present on the container to indicate that the composition is used for a specific therapy or non-therapeutic application, and may also indicate directions for either in vivo or in vitro use, such as those described above.

The 30P3C8 cDNA was deposited under the terms of the Budapest Treaty on Jan. 28, 1999, with the American Type Culture Collection (ATCC; 10801 University Blvd., Manassas, Va. 20110-2209 USA) as plasmid p30P3C8-GTA4, and has been assigned Designation No. 207083.

### EXAMPLES

Various aspects of the invention are further described and illustrated by way of the several examples that follow, none of which are intended to limit the scope of the invention.

### Example 1

### SSH-Generated Isolation of cDNA Fragment of the 30P3C8 Gene

Materials and Methods

LAPC Xenografts

LAPC xenografts were obtained from Dr. Charles Sawyers (UCLA) and generated as described (Klein et al, 1997, Nature Med. 3:402-408; Craft et al., 1999, Cancer Res. 59:5030-5036). Androgen dependent and independent LAPC-4 xenografts (LAPC-4 AD and AI, respectively) were grown in intact male SCID mice or in castrated males, respectively, and were passaged as small tissue chunks in recipient males. LAPC-4 AI xenografts were derived from LAPC-4 AD tumors. To generate the AI xenografts, male mice bearing LAPC AD tumors were castrated and maintained for 2-3 months. After the LAPC tumors re-grew, the tumors were harvested and passaged in castrated males or in female SCID mice.

Cell Lines Human cell lines (e.g., HeLa) were obtained from the ATCC and were maintained in DMEM with 5% fetal calf serum.

RNA Isolation

Tumor tissue and cell lines were homogenized in Trizol reagent (Life Technologies, Gibco BRL) using 10 ml/g tissue or 10 ml/1⁰⁸ cells to isolate total RNA. Poly A RNA was purified from total RNA using Qiagen's Oligotex mRNA Mini and Midi kits. Total and mRNA were quantified by spectrophotometric analysis (O.D. 260/280 nm) and analyzed by gel electrophoresis.

Oligonucleotides

The following HPLC purified oligonucleotides were used.

| | |
|---|---|
| DPNCDN (cDNA synthesis primer): | |
| 5′TTTTGATCAAGCTT₃₀3′ | (SEQ ID NO: 18) |
| Adaptor 1 (SEQ ID NOs: 19, 20, respectively): | |
| 5′CTAATACGACTCACTATAGGGCTCGAGCGGCCGCCCGGGCAG3′ | |
| 3′GGCCCGTCCTAG5′ | |
| Adaptor 2 (SEQ ID NOs: 21, 22, respectively): | |
| 5′GTAATACGACTCACTATAGGGCAGCGTGGTCGCGGCCGAG3′ | |
| 3′CGGCTCCTAG5′ | |
| PCR primer 1: | |
| 5′CTAATACGACTCACTATAGGGC3′ | (SEQ ID NO: 23) |
| Nested primer (NP)1: | |
| 5′TCGAGCGGCCGCCCGGGCAGGA3′ | (SEQ ID NO: 24) |
| Nested primer (NP)2: | |
| 5′AGCGTGGTCGCGGCCGAGGA3′ | (SEQ ID NO: 25) |

Suppression Subtractive Hybridization

Suppression subtractive hybridization (SSH) was used to identify cDNAs corresponding to genes which may be differentially expressed in prostate cancer. The SSH reaction utilized cDNA from two different LAPC xenografts, subtracting LAPC-4 AI cDNA from LAPC-9 AD cDNA. The LAPC-9 AD xenograft was used as the source of the “tester” cDNA, while the LAPC-4 AI cDNA was used as the source of the “driver” cDNA.

Double stranded cDNAs corresponding to tester and driver cDNAs were synthesized from 2 μg of poly(A)+ RNA isolated from the relevant xenograft tissue, as described above, using CLONTECH's PCR-Select cDNA Subtraction Kit and 1 ng of oligonucleotide DPNCDN as primer. First- and second-strand synthesis were carried out as described in the Kit's user manual protocol (CLONTECH Protocol No. PT1117-1, Catalog No. K1804-1). The resulting cDNA was digested with Dpn II for 3 hrs. at 37° C. Digested cDNA was extracted with phenol/chloroform (1:1) and ethanol precipitated.

Driver cDNA was generated by combining in a 1:1 ratio Dpn II digested cDNA from the relevant xenograft source (see above) with a mix of digested cDNAs derived from human benign prostatic hyperplasia (BPH), the human cell lines HeLa, 293, A431, Colo205, and mouse liver.

Tester cDNA was generated by diluting 1 μl of Dpn II digested cDNA from the relevant xenograft source (see above) (400 ng) in 5 μl of water. The diluted cDNA (2 μl, 160 ng) was then ligated to 2 μl of Adaptor 1 and Adaptor 2 (10 μM), in separate ligation reactions, in a total volume of 10 μl at 16° C. overnight, using 400 ug of T4 DNA ligase (CLONTECH). Ligation was terminated with 1 μl of 0.2 M EDTA and heating at 72° C. for 5 min.

The first hybridization was performed by adding 1.5 μl (600 ng) of driver cDNA to each of two tubes containing 1.5 ρl (20 ng) Adaptor 1- and Adaptor 2-ligated tester cDNA. In a final volume of 4 μl, the samples were overlaid with mineral oil, denatured in an MJ Research thermal cycler at 98° C. for 1.5 minutes, and then were allowed to hybridize for 8 hrs at 68° C. The two hybridizations were then mixed together with an additional 1 μl of fresh denatured driver cDNA and were allowed to hybridize overnight at 68° C. The second hybridization was then diluted in 200 μl of 20 mM Hepes, pH 8.3, 50 mM NaCl, 0.2 mM EDTA, heated at 70° C. for 7 min. and stored at −20° C.

PCR Amplification, Cloning and Sequencing of Gene Fragments Generated from SSH:

To amplify gene fragments resulting from SSH reactions, two PCR amplifications were performed. In the primary PCR reaction 1 μl of the diluted final hybridization mix was added to 1 μl of PCR primer 1 (10 μM), 0.5 μl dNTP mix (10 μM), 2.5 μl 10× reaction buffer (CLONTECH) and 0.5 μl 50× Advantage cDNA polymerase Mix (CLONTECH) in a final volume of 25 μl. PCR 1 was conducted using the following conditions: 75° C. for 5 min., 94° C. for 25 sec., then 27 cycles of 94° C. for 10 sec, 66° C. for 30 sec, 72° C. for 1.5 min. Five separate primary PCR reactions were performed for each experiment. The products were pooled and diluted 1:10 with water. For the secondary PCR reaction, 1 μl from the pooled and diluted primary PCR reaction was added to the same reaction mix as used for PCR 1, except that primers NP1 and NP2 (10 μM) were used instead of PCR primer 1. PCR 2 was performed using 10-12 cycles of 94° C. for 10 sec, 68° C. for 30 sec, 72° C. for 1.5 minutes. The PCR products were analyzed using 2% agarose gel electrophoresis.

The PCR products were inserted into pCR2.1 using the T /A vector cloning kit (Invitrogen). Transformed E. coli were subjected to blue/white and ampicillin selection. White colonies were picked and arrayed into 96 well plates and were grown in liquid culture overnight. To identify inserts, PCR amplification was performed on 1 ml of bacterial culture using the conditions of PCR1 and NP1 and NP2 as primers. PCR products were analyzed using 2% agarose gel electrophoresis.

Bacterial clones were stored in 20% glycerol in a 96 well format. Plasmid DNA was prepared, sequenced, and subjected to nucleic acid homology searches of the GenBank, dBest, and NCI-CGAP databases.

RT-PCR Expression Analysis:

First strand cDNAs were generated from 1 μg of mRNA with oligo (dT)12-18 priming using the Gibco-BRL Superscript Preamplification system. The manufacturers protocol was used and included an incubation for 50 min at 42° C. with reverse transcriptase followed by RNAse H treatment at 37° C. for 20 min. After completing the reaction, the volume was increased to 200 μl with water prior to normalization. First strand cDNAs from 16 different normal human tissues were obtained from Clontech.

Normalization of the first strand cDNAs from multiple tissues was performed by using the primers 5′atatcgccgcgctcgtcgtcgacaa3′ (SEQ ID NO: 26) and 5′agccacacgcagctcattgtagaagg 3′ (SEQ ID NO: 27) to amplify β-actin. First strand cDNA (5 μl) was amplified in a total volume of 50 μl containing 0.4 μM primers, 0.2 μM each dNTPs, 1×PCR buffer (Clontech, 10 mM Tris-HCL, 1.5 mM MgCl2, 50 mM KCl, pH8.3) and 1× Klentaq DNA polymerase (Clontech). Five μl of the PCR reaction was removed at 18, 20, and 22 cycles and used for agarose gel electrophoresis. PCR was performed using an MJ Research thermal cycler under the following conditions: initial denaturation was at 94° C. for 15 sec, followed by a 18, 20, and 22 cycles of 94° C. for 15, 65° C. for 2 min, 72° C. for 5 sec. A final extension at 72° C. was carried out for 2 min. After agarose gel electrophoresis, the band intensities of the 283 bp β-actin bands from multiple tissues were compared by visual inspection. Dilution factors for the first strand cDNAs were calculated to result in equal β-actin band intensities in all tissues after 22 cycles of PCR. Three rounds of normalization were required to achieve equal band intensities in all tissues after 22 cycles of PCR.

To determine expression levels of the 30P3C8 gene, 5 μl of normalized first strand cDNA was analyzed by PCR using 25, 30, and 35 cycles of amplification using the following primer pairs, which were designed with the assistance of (MIT; for details, see, www.genome.wi.mit.edu):

| | |
|---|---|
| 5′-TGT ACA CAT TTA GCT TGT GGT-3′ | (SEQ ID NO: 28) |
| 5′-GCC AGT TAT TTG CAA GTG GTA | (SEQ ID NO: 29) |
| AGA G-3′ | |

Semi quantitative expression analysis was achieved by comparing the PCR products at cycle numbers that give light band intensities.

Results

The SSH experiments described in the Materials and Methods, supra, led to the isolation of numerous candidate gene fragment clones (SSH clones). All candidate clones were sequenced and subjected to homology analysis against all sequences in the major public gene and EST databases in order to provide information on the identity of the corresponding gene and to help guide the decision to analyze a particular gene for differential expression. In general, gene fragments which had no homology to any known sequence in any of the searched databases, and thus considered to represent novel genes, as well as gene fragments showing homology to previously sequenced expressed sequence tags (ESTs), were subjected to differential expression analysis by RT-PCR and/or Northern analysis.

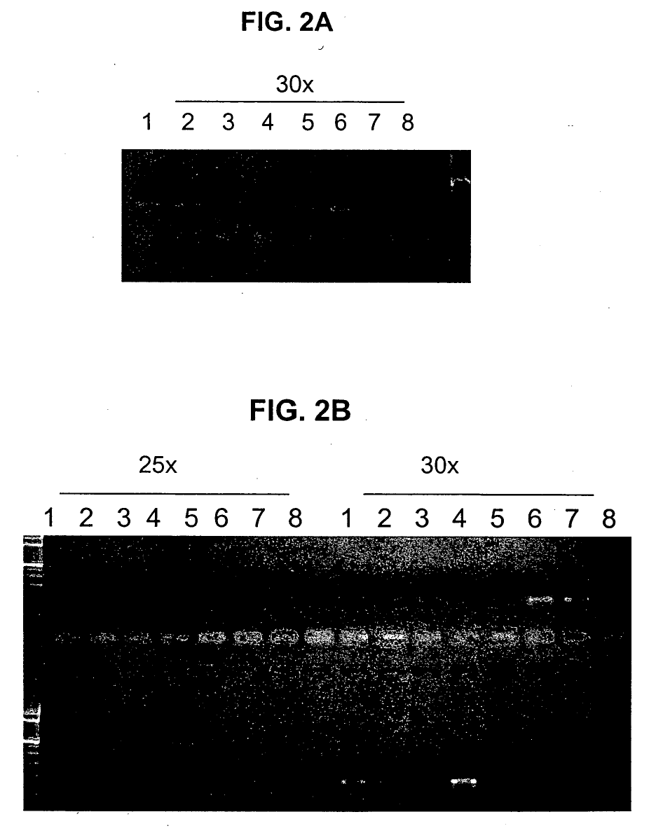

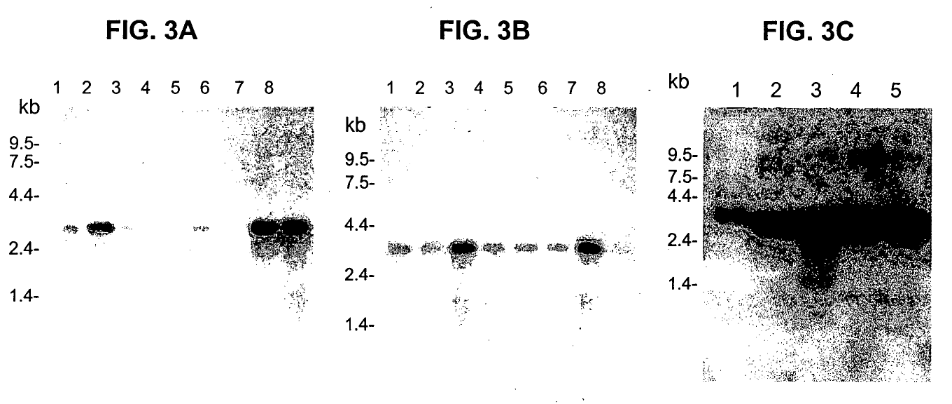

One of the SHH clones, comprising about 362 bp, exhibits significant homology to ESTs derived from several libraries, including libraries generated from testis, parathyroid tumor, fetal heart and kidney. This SSH clone, designated 30P3C8, was used to design primers for RT-PCR expression analysis of the 30P3C8 gene in various tissues. RT-PCR analysis showed that 30P3C8 is expressed in prostate, brain and all the LAPC xenografts analyzed (FIG. 2A). RT-PCR analysis of first strand cDNA derived from 16 normal tissues showed expression primarily in prostate and placenta after 25 cycles of amplification, although lower level expression is detected in other tissues after 30 cycles of amplification (FIG. 2B). Northern blot analysis using the 30P3C8 SSH fragment as probe shows over-expression of 30P3C8 in prostate cancer xenografts (FIGS. 3A-3C).

### Example 2

### Cloning of Full Length 30P3C8 cDNA

A full length cDNA encoding the 30P3C8 gene was isolated from a human prostate library and designated 30P3C8-GTA4. The nucleotide and amino acid sequences of 30P3C8-GTA4 are shown in FIG. 1. Plasmid p30P3C8-GTA4 (carrying the 30P3C8-GTA4 cDNA) was deposited with the ATCC (Manassas, Va.) on January 28, 1999 and has been accorded ATCC Designation Number 207083. The approximately 3 kb 30P3C8-GTA4 cDNA encodes a protein of 400 or 401 amino acids containing an N-terminal signal sequence and a putative cleavage site at amino acid residue 28 or 29. Computer analysis of this sequence predicts that 30P3C8 is a secreted protein. In addition, the 5′ untranslated region of the 30P3C8 transcript is very GC rich (>75%), suggesting possible translational regulation of 30P3C8. The 30P3C8 cDNA sequence shows significant homology to a number of ESTs derived from a variety of sources, including testis, parathyroid tumor, fetal heart and kidney libraries. However, the 30P3C8 cDNA does not show any significant homology to any known gene.

### Example 3

### 30P3C8 Gene Expression Analysis

To analyze 30P3C8 expression in cancer tissues, northern blotting was performed on RNA derived from the LAPC xenografts, and several prostate and non-prostate cancer cell lines. The results show very high expression levels in LAPC-4 AD, LAPC-4 AI, LAPC-9 AD, LAPC-9 AI (FIG. 4A) and lower expression in LAPC-3 AI (FIG. 5). More detailed analysis of the xenografts shows that 30P3C8 is highly expressed in the xenografts even when grown within the tibia of mice (FIG. 5).

High expression levels of 30P3C8 were detected in several cancer cell lines derived from prostate (LNCaP, DU145, LAPC-4CL), pancreas (HPAC, Capan-1), colon (SK-CO-1, CaCo-2, LoVo, T84, Colo-205), brain (PFSK-1, T98G), bone (SK-ES-1, HOS, U2-OS, RD-ES), lung (CALU-1, A427, NCI-H82, NCI-H146) and kidney (769-P, A498, CAKI-1, SW839) (FIGS. 4A-4B). Lower expression levels were also detected in multiple bladder, pancreatic and prostate cancer cell lines. Northern analysis also shows that 30P3C8 is expressed at high levels in the normal prostate and prostate tumor tissues derived from prostate cancer patients (FIG. 6A).

### Example 4

### Secretion of 30P3C8 in vitro

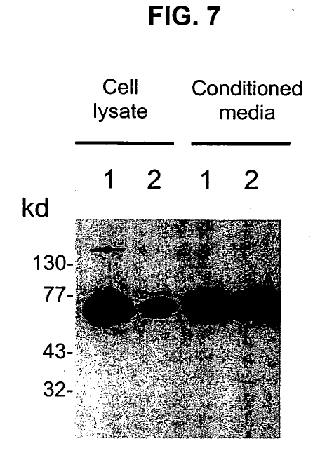

To demonstrate that 30P3C8 protein is indeed secreted, the 30P3C8 ORF sequence (FIGS. 1A-1D (SEQ ID NO: 1)) was inserted into pCDNA 3.1 myc-his (Invitrogen), which provides a carboxyl-terminal myc-his tag. Forty-eight hours after transfection into 293T cells, the conditioned media was collected and cell lysates were prepared. His-tagged 30P3C8 protein was purified using a Nickel column, which has a high affinity for His tags. Protein was visualized by western blotting using anti-His tag antibodies. The results from duplicate experiments clearly show that 30P3C8 protein is present in cell lysates as well as in conditioned media (FIG. 7), indicating that the 30P3C8 protein is secreted.

### Example 5

### Generation of 30P3C8 Polyclonal Antibodies and Detection of 30P3C8 in Prostate Cancer Patient Tissues, Cell Lines and Supernatant

To generate polyclonal sera to 30P3C8 a peptide was synthesized corresponding to amino acids 375-389 PVFNVEDQKRDTINL; SEQ ID NO: 30) of the 30P3C8 protein sequence. This peptide was coupled to Keyhole limpet hemacyanin (KLH) and used to immunize a rabbit as follows. The rabbit was initially immunized with 200 μg of peptide-KLH mixed in complete Freund's adjuvant. The rabbit was then injected every two weeks with 200 μg of peptide-KLH in incomplete Freund's adjuvant. Bleeds were taken approximately 7-10 days following each immunization. ELISA and western blotting analyses were used to determine titer and specificity of the rabbit serum to the immunizing peptide and to 30P3C8 protein respectively. Affinity purified anti-30P3C8 polyclonal antibodies were prepared by passage of crude serum from immunized rabbit over an affinity matrix comprised of 30P3C8 peptide covalently coupled to Affigel 15 (BioRad). After extensive washing of the matrix with PBS, antibodies specific to 30P3C8 peptide were eluted with low pH glycine buffer (0.1M, pH 2.5) and dialyzed against PBS.

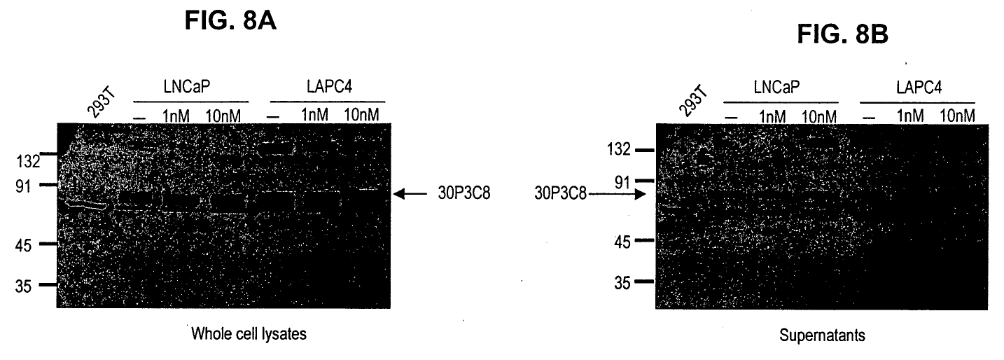

LNCaP and LAPC4 cell lines were starved of androgen by incubation of cells in 2% charcoal-dextran stripped FBS for 4 days and then incubated with or without either 1 or 10 nM of the androgen analog mibolerone for 48 hours and then cells and conditioned supernatants were harvested. Cell lysates (made in 2x SDS-PAGE sample buffer) and conditioned media (0.22 μM filtered) were then subjected to western analysis with an affinity purified rabbit anti-peptide pAb raised to amino acids 375-389 of 30P3C8 (VFNVEDQKRDTINL; SEQ ID NO: 30). Cell lysates (25 μg/lane) and supernatants (20 μl) from LNCaP and LAPC4 cells or from 293T cells as a negative control were separated by 10-20% gradient SDS-PAGE transferred to nitrocellulose and subjected to western analysis using 2 μg/ml of affinity purified anti-30P3C8 pAb. Anti-30P3C8 immunoreactive bands were visualized by incubation with anti-rabbit-HRP conjugated secondary antibody and enhanced chemiluminescence detection (FIGS. 8A-8B).

The first 28 amino acids of 30P3C8 contains a predicted signal peptide that suggests that 30P3C8 is a secreted protein. The anti-30P3C8 western analysis of LAPC4 and LNCAP prostate cancer cell lines and conditioned media derived from these cell lines demonstrates the presence of specific 85 kD 30P3C8 immunoreactive band in both whole cell lysates and supernatants (FIGS. 8A-8B). This suggests that 30P3C8 is a secreted protein and a potential diagnostic marker of prostate cancer. The amount of 30P3C8 protein did not vary significantly in androgen starved or stimulated LAPC4 and LNCAP cells suggesting that its expression is not tightly androgen regulated.

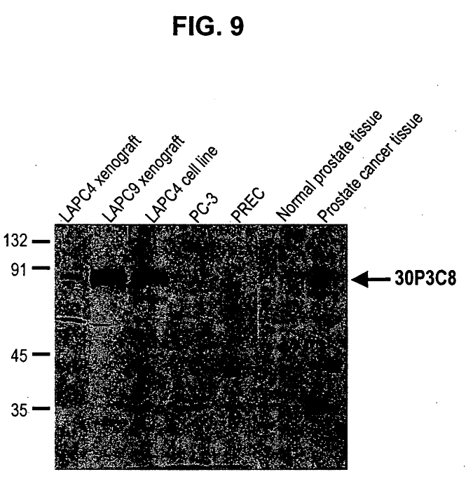

Tissue lysates representing LAPC4 and LAPC9 xenografts, clinical biopsy specimens representing matched normal adjacent tissue and prostate cancer tissues, whole cell lysates of LAPC4 cells, PC3 cells (androgen receptor negative), and normal prostate epithelial cells (Clonetics) were subjected to western analysis using affinity purified anti-30P3C8 pAb as described above. 30P3C8 protein appears to be upregulated in prostate cancer tissue inasmuch as expression is seen in LAPC4 and LAPC9 xenografts and a prostate cancer tissue biopsy specimen, but is not detected in a matched normal prostate tissue biopsy or in normal prostate epithelial cells or in the androgen receptor negative prostate cancer cell line PC3 (FIG. 9). The predicted MW of 30P3C8 based on its amino acid sequence is 45.2 kD thus the presence of a specific 85 kD immunoreactive band in western analysis suggests that 30P3C8 may undergo extensive post-translational modifications or possibly exist as a dimer that is resistant to SDS and heat denaturation.

### Example 6

### Production of Recombinant 30P3C8 in a Mammalian System

To express recombinant 30P3C8, the full length 30P3C8 cDNA can be cloned into an expression vector that provides a 6His tag at the carboxyl-terminus (PCDNA 3.1 myc-his, Invitrogen). The constructs can be transfected into 293T cells. Transfected 293T cell lysates can be probed with the anti-30P3C8 polyclonal serum described in Example 5 above in a western blot.

The 30P3C8 genes can also be subcloned into the retroviral expression vector pSRaMSVtkneo and used to establish 30P3C8 expressing cell lines as follows. The 30P3C8 coding sequence (from translation initiation ATG to the termination codons) is amplified by PCR using ds cDNA template from 30P3C8 cDNA. The PCR product is subcloned into pSRαMSVtkneo via the EcoR1(blunt-ended) and Xba 1 restriction sites on the vector and transformed into DH5α competent cells. Colonies are picked to screen for clones with unique internal restriction sites on the cDNA. The positive clone is confirmed by sequencing of the cDNA insert. Retroviruses may thereafter be used for infection and generation of various cell lines using, for example, NIH 3T3, TsuPr1, 293 or rat-1 cells.

### Example 7

### Production of Recombinant 30P3C8 in a Baculovirus System

To generate a recombinant 30P3C8 protein in a baculovirus expression system, the 30P3C8 cDNA is cloned into the baculovirus transfer vector pBlueBac 4.5 (Invitrogen), which provides a His-tag at the N-terminus Specifically, pBlueBac-30P3C8 is co-transfected with helper plasmid pBac-N-Blue (Invitrogen) into SF9 (Spodoptera frugiperda) insect cells to generate recombinant baculovirus (see Invitrogen instruction manual for details). Baculovirus is then collected from cell supernatant and purified by plaque assay.

Recombinant 30P3C8 protein is then generated by infection of HighFive insect cells (Invitrogen) with the purified baculovirus. Recombinant 30P3C8 protein may be detected using anti-30P3C8 antibody. 30P3C8 protein may be purified and used in various cell based assays or as immunogen to generate polyclonal and monoclonal antibodies specific for 30P3C8.

### Example 8

### Identification of Potential Signal Transduction Pathways

To determine whether 30P3C8 directly or indirectly activates known signal transduction pathways in cells, luciferase (luc) based transcriptional reporter assays are carried out in cells expressing 30P3C8. These transcriptional reporters contain consensus binding sites for known transcription factors that lie downstream of well characterized signal transduction pathways. The reporters and examples of their associated transcription factors, signal transduction pathways, and activation stimuli are listed below.

1. NFkB-luc, NFkB/Rel; Ik-kinase/SAPK; growth/apoptosis/stress

2. SRE-luc, SRF/TCF/ELK1; MAPK/SAPK; growth/differentiation

3. AP-1-luc, FOS/JUN; MAPK/SAPK/PKC; growth/apoptosis/stress

4. ARE-luc, androgen receptor; steroids/MAPK; growth/differentiation/apoptosis

5. p53-luc, p53; SAPK; growth/differentiation/apoptosis

6. CRE-luc, CREB/ATF2; PKA/p38; growth/apoptosis/stress

30P3C8-mediated effects may be assayed in cells showing mRNA expression. Luciferase reporter plasmids may be introduced by lipid mediated transfection (TFX-50, Promega). Luciferase activity, an indicator of relative transcriptional activity, is measured by incubation of cells extracts with luciferin substrate and luminescence of the reaction is monitored in a luminometer.

### Example 9

### Generation of 30P3C8 Monoclonal Antibodies

In order to generate 30P3C8 monoclonal antibodies, a glutathione-S-transferase (GST) fusion protein encompassing a 30P3C8 protein is synthesized and used as immunogen. Balb C mice are initially immunized intraperitoneally with 200 μg of the GST-30P3C8 fusion protein mixed in complete Freund's adjuvant. Mice are subsequently immunized every 2 weeks with 75 μg of GST-30P3C8 protein mixed in Freund's incomplete adjuvant for a total of 3 immunizations. Reactivity of serum from immunized mice to full length 30P3C8 protein is monitored by ELISA using a partially purified preparation of HIS-tagged 30P3C8 protein expressed from 293T cells (Example 6). Mice showing the strongest reactivity are rested for 3 weeks and given a final injection of fusion protein in PBS and then sacrificed 4 days later. The spleens of the sacrificed mice are then harvested and fused to SPO/2 myeloma cells using standard procedures (Harlow and Lane, 1988, supra). Supernatants from growth wells following HAT selection are screened by ELISA and western blot to identify 30P3C8 specific antibody producing clones.

The binding affinity of a 30P3C8 monoclonal antibody may be determined using standard technology. Affinity measurements quantify the strength of antibody to epitope binding and may be used to help define which 30P3C8 monoclonal antibodies are preferred for diagnostic or therapeutic use. The BIAcore system (Uppsala, Sweden) is a preferred method for determining binding affinity. The BIAcore system uses surface plasmon resonance (SPR, Welford, K., 1991, Opt. Quant. Elect. 23:1; Morton and Myszka, 1998, Methods in Enzymology 295:268) to monitor biomolecular interactions in real time. BIAcore analysis conveniently generates association rate constants, dissociation rate constants, equilibrium dissociation constants, and affinity constants.

### Example 10

### In Vitro Assays of 30P3C8 Function

The expression of 30P3C8 in prostate cancer provides evidence that this gene has a functional role in tumor progression. It is possible that 30P3C8 functions as a secreted protein involved in activating signals involved in tumorigenesis and/or tumor growth. 30P3C8 function can be assessed in mammalian cells using in vitro approaches.

For mammalian expression, 30P3C8 can be cloned into a number of appropriate vectors, including pcDNA 3.1 myc-His-tag (Example 6) and the retroviral vector pSRoxtkneo (Muller et al., 1991, MCB 11:1785). Using such expression vectors, 30P3C8 can be expressed in several cell lines, including NIH 3T3, rat-1, TsuPr1 and 293T. Expression of 30P3C8 can be monitored using anti-30P3C8 antibodies (see Examples 5 and 9).

Mammalian cell lines expressing 30P3C8 can be tested in several in vitro and in vivo assays, including cell proliferation in tissue culture, activation of apoptotic signals, tumor formation in SCID mice, and in vitro invasion using a membrane invasion culture system (MICS) (Welch et al., 1989, Int. J. Cancer 43:449-457). 30P3C8 cell phenotype is compared to the phenotype of cells that lack expression of 30P3C8.

Cell lines expressing 30P3C8 can also be assayed for alteration of invasive and migratory properties by measuring passage of cells through a matrigel coated porous membrane chamber (Becton Dickinson). Passage of cells through the membrane to the opposite side is monitored using a fluorescent assay (Becton Dickinson Technical Bulletin #428) using calcein-Am (Molecular Probes) loaded indicator cells. Cell lines analyzed include parental and 30P3C8 overexpressing PC3, NIH 3T3 and LNCaP cells. To determine whether 30P3C8-expressing cells have chemoattractant properties, indicator cells are monitored for passage through the porous membrane toward a gradient of 30P3C8 conditioned media compared to control media. This assay may also be used to qualify and quantify specific neutralization of the 30P3C8 induced effect by candidate cancer therapeutic compositions.

The function of 30P3C8 can be evaluated using anti-sense RNA technology coupled to the various functional assays described above, e.g. growth, invasion and migration. Anti-sense RNA oligonucleotides can be introduced into 30P3C8 expressing cells, thereby preventing the expression of 30P3C8. Control and anti-sense containing cells can be analyzed for proliferation, invasion, migration, apoptotic and transcriptional potential. The local as well as systemic effect of the loss of 30P3C8 expression can be evaluated.

### Example 11

### In Vivo Assay for 30P3C8 Tumor Growth Promotion

The effect of the 30P3C8 protein on tumor cell growth may be evaluated in vivo by gene overexpression in tumor-bearing mice. For example, SCID mice can be injected subcutaneously on each flank with 1×10⁶ of either PC3, TSUPR1, or DU145 cells containing tkNeo empty vector or 30P3C8. At least two strategies may be used: (1) Constitutive 30P3C8 expression under regulation of a promoter such as a constitutive promoter obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), or from heterologous mammalian promoters, e.g., the actin promoter or an immunoglobulin promoter, provided such promoters are compatible with the host cell systems, and (2) Regulated expression under control of an inducible vector system, such as ecdysone, tet, etc., provided such promoters are compatible with the host cell systems. Tumor volume is then monitored at the appearance of palpable tumors and followed over time to determine if 30P3C8 expressing cells grow at a faster rate and whether tumors produced by 20P2H8-expressing cells demonstrate characteristics of altered aggressiveness (e.g. enhanced metastasis, vascularization, reduced responsiveness to chemotherapeutic drugs). Additionally, mice may be implanted with 1×10⁵ of the same cells orthotopically to determine if 30P3C8 has an effect on local growth in the prostate or on the ability of the cells to metastasize, specifically to lungs, lymph nodes, and bone marrow.

The assay is also useful to determine the 30P3C8 inhibitory effect of candidate therapeutic compositions, such as for example, 30P3C8 intrabodies, 30P3C8 antisense molecules and ribozymes.

### Example 12

### Western Analysis of 30P3C8 Expression in Subcellular Fractions

Sequence analysis of 30P3C8 revealed the presence of a secretion signal sequence. The cellular location of 30P3C8 can be assessed using subcellular fractionation techniques widely used in cellular biology (Storrie B, et al., 1990, Methods Enzymol. 182:203-25). Prostate or other cell lines can be separated into cell supernatant, nuclear, cytosolic and membrane fractions. The expression of 30P3C8 in the different fractions can be tested using western blotting techniques.

Alternatively, to determine the subcellular localization of 30P3C8, 293T cells can be transfected with an expression vector encoding HIS-tagged 30P3C8 (PCDNA 3.1 MYC/HIS, Invitrogen). The transfected cells can be harvested and subjected to a differential subcellular fractionation protocol as previously described (Pemberton, P. A. et al., 1997, J. Histochem. Cytochem. 45:1697-1706.) This protocol separates the cell into fractions enriched for nuclei, heavy membranes (lysosomes, peroxisomes, and mitochondria), light membranes (plasma membrane and endoplasmic reticulum), and soluble proteins.

Throughout this application, various publications are referenced within parentheses. The disclosures of these publications are hereby incorporated by reference herein in their entireties.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

1-37. (Cancelled)

38. A vaccine composition for the treatment of a cancer expressing 30P3C8 comprising an immunogenic portion of a 30P3C8 polypeptide and a physiologically acceptable carrier.

39. A method of inhibiting the development of a cancer expressing 30P3C8 in a patient, comprising administering to the patient effective amount of the vaccine composition of claim 38.


SEQUENCE LISTING (30 sequences)

SEQ ID NO: 1 - DNA, 3051 bp, Homo sapien
  CDS (163)...(1365)
gacgcgtgggcgcggaggcgctgggcgcacggcgcggagccggccggagctcgaggccggcggcggcgggagagcgacccgggcggcctcgtagcggggccccggatccccgagtggcggccggagcctcgaaaagagattctcagcgctgattttgagatgatgggcttgggaaacgggcgtcgcagcatgaagtcgccgcccctcgtgctggccgccctggtggcctgcatcatcgtcttgggcttcaactactggattgcgagctcccggagcgtggacctccagacacggatcatggagctggaaggcagggtccgcagggcggctgcagagagaggcgccgtggagctgaagaagaacgagttccagggagagctggagaagcagcgggagcagcttgacaaaatccagtccagccacaacttccagctggagagcgtcaacaagctgtaccaggacgaaaaggcggttttggtgaataacatcaccacaggtgagaggctcatccgagtgctgcaagaccagttaaagaccctgcagaggaattacggcaggctgcagcaggatgtcctccagtttcagaagaaccagaccaacctggagaggaagttctcctacgacctgagccagtgcatcaatcagatgaaggaggtgaaggaacagtgtgaggagcgaatagaagaggtcaccaaaaaggggaatgaagctgtagcttccagagacctgagtgaaaacaacgaccagagacagcagctccaagccctcagtgagcctcagcccaggctgcaggcagcaggcctgccacacacagaggtgccacaagggaagggaaacgtgcttggtaacagcaagtcccagacaccagcccccagttccgaagtggttttggattcaaagagacaagttgagaaagaggaaaccaatgagatccaggtggtgaatgaggagcctcagagggacaggctgccgcaggagccaggccgggagcaggtggtggaagacagacctgtaggtggaagaggcttcgggggagccggagaactgggccagaccccacaggtgcaggctgccctgtcagtgagccaggaaaatccagagatggagggccctgagcgagaccagcttgtcatccccgacggacaggaggaggagcaggaagctgccggggaagggagaaaccagcagaaactgagaggagaagatgactacaacatggatgaaaatgaagcagaatctgagacagacaagcaagcagccctggcagggaatgacagaaacatagatgtttttaatgttgaagatcagaaaagagacaccataaatttacttgatcagcgtgaaaagcggaatcatacactctgaattgaactggaatcacatatttcacaacagggccgaagagatgactataaaatgttcatgagggactgaatactgaaaactgtgaaatgtactaaataaaatgtacatctgaagatgattattgtgaaattttagtatgcactttgtgtaggaaaaaatggaatggtcttttaaacagcttttggggggtactttggaagtgtctaataaggtgtcacaatttttggtagtaggtatttcgtgagaagttcaacaccaaaactggaacatagttctccttcaagtgttggcgacagcggggcttcctgattctggaatataactttgtgtaaattaacagccacctatagaagagtccatctgctgtgaaggagagacagagaactctgggttccgtcgtcctgtccacgtgctgtaccaagtgctggtgccagcctgttacctgttctcactgaaaagtctggctaatgctcttgtgtagtcacttctgattctgacaatcaatcaatcaatggcctagagcactgactgttaacacaaacgtcactagcaaagtagcaacagctttaagtctaaatacaaagctgttctgtgtgagaattttttaaaaggctacttgtataataacccttgtcatttttaatgtacaaaacgctattaagtggcttagaatttgaacatttgtggtctttatttactttgcttcgtgtgtgggcaaagcaacatcttccctaaatatatattaccaagaaaagcaagaagcagattaggtttttgacaaaacaaacaggccaaaagggggctgacctggagcagagcatggtgagaggcaaggcatgagagggcaagtttgttgtggacagatctgtgcctactttattactggagtaaaagaaaacaaagttcattgatgtcgaaggatatatacagtgttagaaattaggactgtttagaaaaacaggaatacaatggttgtttttatcatagtgtacacatttagcttgtggtaaatgactcacaaaactgattttaaaatcaagttaatgtgaattttgaaaattactacttaatcctaattcacaataacaatggcattaaggtttgacttgagttggttcttagtattatttatggtaaataggctcttaccacttgcaaataactggccacatcattaatgactgacttcccagtaaggctctctaaggggtaagtaggaggatccacaggatttgagatgctaaggccccagagatcgtttgatccaaccctcttattttcagaggggaaaatggggcctagaagttacagagcatctagctggtgcgctggcacccctggcctcacacagactcccgagtagctgggactacaggcacacagtcactgaagcaggccctgtttgcaattcacgttgccacctccaacttaaacattcttcatatgtgatgtccttagtcactaaggttaaactttcccacccagaaaaggcaacttagataaaatcttagagtactttcatactcttctaagtcctcttccagcctcactttgagtcctccttggggttgataggaattttctcttgctttctcaataaagtctctattcatctcatgtttaatttgtacgcatagaattgctgagaaataaaatgttctgttcaacttaaaaaaaaaaaaaaaa

SEQ ID NO: 2 - protein, 400 aa, Homo sapien [sequence omitted]

SEQ ID NO: 3 - protein, 4 aa, Homo sapien
NITT

SEQ ID NO: 4 - protein, 4 aa, Homo sapien
NQTN

SEQ ID NO: 5 - protein, 4 aa, Homo sapien
NHTL

SEQ ID NO: 6 - protein, 4 aa, Homo sapien
RKFS

SEQ ID NO: 7 - protein, 4 aa, Homo sapien
KRDT

SEQ ID NO: 8 - protein, 4 aa, Homo sapien
TTGE

SEQ ID NO: 9 - protein, 4 aa, Homo sapien
TNLE

SEQ ID NO: 10 - protein, 4 aa, Homo sapien
SETD

SEQ ID NO: 11 - protein, 8 aa, Homo sapien
KLRGEDDY

SEQ ID NO: 12 - protein, 6 aa, Homo sapien
GLGNGR

SEQ ID NO: 13 - protein, 6 aa, Homo sapien
GLPHTE

SEQ ID NO: 14 - protein, 6 aa, Homo sapien
GNVLGN

SEQ ID NO: 15 - protein, 6 aa, Homo sapien
GNSKSQ

SEQ ID NO: 16 - protein, 6 aa, Homo sapien
GNDRNI

SEQ ID NO: 17 - protein, 4 aa, Homo sapien
NGRR

SEQ ID NO: 18 - DNA, 14 bp, Homo sapien
ttttgatcaagctt

SEQ ID NO: 19 - DNA, 42 bp, Homo sapien
ctaatacgactcactatagggctcgagcggccgcccgggcag

SEQ ID NO: 20 - DNA, 12 bp, Homo sapien
ggcccgtcctag

SEQ ID NO: 21 - DNA, 40 bp, Homo sapien
gtaatacgactcactatagggcagcgtggtcgcggccgag

SEQ ID NO: 22 - DNA, 10 bp, Homo sapien
cggctcctag

SEQ ID NO: 23 - DNA, 22 bp, Homo sapien
ctaatacgactcactatagggc

SEQ ID NO: 24 - DNA, 22 bp, Homo sapien
tcgagcggccgcccgggcagga

SEQ ID NO: 25 - DNA, 20 bp, Homo sapien
agcgtggtcgcggccgagga

SEQ ID NO: 26 - DNA, 25 bp, Homo sapien
atatcgccgcgctcgtcgtcgacaa

SEQ ID NO: 27 - DNA, 26 bp, Homo sapien
agccacacgcagctcattgtagaagg

SEQ ID NO: 28 - DNA, 21 bp, Homo sapien
tgtacacatttagcttgtggt

SEQ ID NO: 29 - DNA, 25 bp, Homo sapien
gccagttatttgcaagtggtaagag

SEQ ID NO: 30 - protein, 15 aa, Homo sapien
DVFNVEDQKRDTINL